(19) United States
(12) Patent Application Publication
(10) Pub. No.: US 2016/0257728 A1
(43) Pub. Date: Sep. 8, 2016

(54) BIOACTIVE RECOMBINANT BMP-9 PROTEIN, MB109, EXPRESSED AND ISOLATED FROM BACTERIA

(71) Applicant: JOINT CENTER FOR BIOSCIENCES, Yeonsu-ku, Incheon (KR)

(72) Inventors: Senyon Choe, Solana Beach, CA (US); Mario Meng-Chiang Kuo, San Diego, CA (US); Jae Woo Jung, Seoul (KR); Dong Kun Lee, Seoul (KR)

(21) Appl. No.: 15/029,610

(22) PCT Filed: Oct. 16, 2014
(86) PCT No.: PCT/IB2014/002443

§ 371 Date: Apr. 14, 2016

Related U.S. Application Data
(60) Provisional application No. 61/892,096, filed on Oct. 17, 2013.

(51) Int. Cl. C07K 14/51

(52) U.S. Cl. CPC C07K 14/51; A61K 38/00

(57) ABSTRACT

The disclosure provides for the expression and purification of bioactive recombinant bone morphogenetic protein (BMP)-9 from bacteria (MB109) and the use of the MB109 to treat various diseases and disorders, including cancer and obesity associated disorders.

### CROSS REFERENCE TO RELATED APPLICATIONS

This application claims priority under 35 U.S.C. §119 from Provisional Application Ser. No. 61/892,096, filed Oct. 17, 2013, the disclosure of which is incorporated herein by reference.

### TECHNICAL FIELD

The disclosure provides for the expression and purification of bioactive recombinant bone morphogenetic protein-9 from bacteria (MB109) and the use of the MB109 protein to treat various diseases, disorders, and conditions.

### BACKGROUND

BMP-9 has been distinguished from other BMPs by its unique receptor-binding specificity and its diverse roles in a variety of cellular processes. For example, BMP-9 is able to inhibit the production of hepatic glucose and to activate the expression of several key enzymes in lipid metabolism; regulate the growth and migration of endothelial cells; induce apoptosis in prostate cancer cells and to promote the proliferation of ovarian cancer cells; and is one of the most potent BMPs to induce osteogenic differentiation and orthotopic bone formation. Due to the complex disulfide linking feature, production of bioactive polypeptides of recombinant human BMP-9 has only been achieved by using the mammalian Chinese hamster ovary (CHO) cells.

### SUMMARY

The disclosure provides for the expression and purification of a bioactive recombinant polypeptide from bacteria, which is referred to herein as MB109, that has BMP-9 protein activity. The disclosure also provides for treating a variety of conditions, diseases and disorders, such as cancer and diabetes, with the bioactive MB109 protein disclosed herein.

The disclosure provides a non-naturally occurring recombinant protein comprising two or more polypeptides having least 90% sequence identity to SEQ ID NO:2 with a methionine at position 1, wherein the protein has similar bioactivity to human bone morphogenetic protein-9 (human BMP-9). In one embodiment, the protein comprises a homodimer of two polypeptides each polypeptide comprising a sequence of SEQ ID NO:2, wherein the homodimer comprises a cysteine knot scaffold formed from three intra-molecular and one inter-molecular disulfide bonds. In another embodiment of either of the foregoing the protein is expressed and isolated from bacteria selected from the group consisting of Escherichia Coli, Corynebacterium glutamicum, and Pseudomonas fluorescens. In yet another embodiment of the foregoing the two or more polypeptides are encoded by a Escherichia Coli, Corynebacterium glutamicum, or Pseudomonas fluorescens codon optimized polynucleotide sequence having at least 85% sequence identity to SEQ ID NO:1 and encoding a polypeptide of SEQ ID NO:2. In another embodiment, the two or more polypeptides are encoded by a Escherichia Coli codon optimized polynucleotide sequence comprising SEQ ID NO:1. In yet another embodiment of any of the foregoing, the protein has been isolated and refolded from a bacterial inclusion body. In yet a further embodiment, the bacterial inclusion body is from Escherichia Coli. In yet another embodiment, the protein has been purified to remove bacterial host cell contaminants. In yet any of the foregoing embodiments, the protein has been re-folded using one or more of the following folding conditions: a buffer pH between about 8.0 and 8.5; a CHAPS concentration between about 2 and 4%; a NaCl concentration between about 1 and 2M; a redox system having a GSH/GSSG molar ratio between about 5:1 and 1:2; a protein concentration of about 0.2 mg/mL or lower; and a refolding temperature/duration of about 4° C. for around 7 to 9 days.

The disclosure also provides a bioactive recombinant polypeptide (MB109) having BMP-9 activity expressed and isolated from bacteria. In one embodiment, the polypeptide comprises a multimer of polypeptides of SEQ ID NO:2. In yet another embodiment, the bacteria is selected from group consisting of Escherichia Coli, Corynebacterium glutamicum, or Pseudomonas fluorescens. In yet another embodiment, the recombinant MB109 is expressed and isolated from an Escherichia Coli inclusion body. In another embodiment of either of the foregoing, the MB109 polypeptide is encoded by a polynucleotide comprising at least 85% sequence identity to SEQ ID NO:1 and which encodes a polypeptide of SEQ ID NO:2. In another embodiment, the MB109 polypeptide is encoded by a Escherichia Coli codon optimized polynucleotide sequence comprising SEQ ID NO:1. In yet another embodiment, the MB109 has been re-folded using one or more of the following folding conditions: a buffer pH between about 8.0 and 8.5; a CHAPS concentration between about 2 and 4%; a NaCl concentration between about 1 and 2M; a redox system having a GSH/GSSG molar ratio between about 5:1 and 1:2; a proteinaceous concentration of about 0.2 mg/mL or lower; and a refolding temperature/duration of about 4° C. for around 7 to 9 days.

The disclosure also provides a pharmaceutical composition comprising the MB109 polypeptide as described in any of the embodiments above in combination with a pharmaceutically acceptable carrier.

The disclosure also provides a method of treating cancer in a subject comprising administering an MB109 polypeptide or pharmaceutical composition as described above to a subject in need of such treatment. In one embodiment, the cancer is selected from the group consisting of adrenocortical carcinoma, anal cancer, bladder cancer, brain tumors, and ependymomas, breast cancer, gastrointestinal carcinoid tumors, cervical cancer, colon cancer, endometrial cancer, esophageal cancer, extrahepatic bile duct cancer, ewings family of tumors (PNET), extracranial germ cell tumors, extragonadal germ cell tumors, eye cancer, including intraocular melanomas, gallbladder cancer, gastric cancer (stomach), gestational trophoblastic tumor, head and neck cancer, hypopharyngeal cancer, islet cell carcinoma, kidney Cancer (renal cell cancer), laryngeal cancer, leukemias, lip and oral cavity cancer, liver cancer, lung cancer, lymphomas, malignant mesothelioma, melanoma, merkel cell carcinoma, metasatic squamous neck cancer with occult primary, multiple myeloma and other plasma cell neoplasms, mycosis fungoides, myelodysplastic syndrome, myeloproliferative disorders, nasopharyngeal cancer, neuroblastoma, oral cancer, oropharyngeal cancer, osteosarcoma, ovarian epithelial cancer, ovarian germ cell tumor, pancreatic cancers, paranasal sinus and nasal cavity cancer, parathyroid cancer, penile cancer, pituitary cancer, plasma cell neoplasm, prostate cancer, rhabdomyosarcoma, rectal cancer, renal cell cancer (cancer of the kidney), transitional cell renal pelvis and ureter cancer, salivary gland cancer, sezary syndrome, skin cancers, small intestine cancer, soft tissue sarcoma, stomach cancer, testicular cancer, malignant thyoma, thyroid cancer, urethral cancer, uterine cancer, vaginal cancer, vulvar cancer, and Wilms' tumor. In a specific embodiment, the cancer is liver cancer. In yet a further embodiment, the liver cancer is a hepatocellular carcinoma. The method can further include administering one or more additional pharmaceutically active agents. For example, the additional pharmaceutically active agents can be a chemotherapeutic and/or anti-cancer agents.

The disclosure also provides a method of treating obesity and/or an obesity associated disorder in a subject comprising administering the protein or the pharmaceutical composition as described in any of the foregoing embodiments to a subject in need of such therapy. In one embodiment, the obesity associated disorder is selected from the group consisting of type 2 diabetes, heart disease, stroke, hypertension, liver disease, gallbladder disease, osteoarthritis, metabolic syndrome, polycystic ovary syndrome (PCOS), reproductive hormonal abnormalities, and dyslipidemia. In a specific embodiment, the obesity related disorder is type 2 diabetes. In yet a further embodiment, the method can further include administering one or more additional pharmaceutically active agents. In yet a further embodiment, the additional pharmaceutically active agents are selected from the group consisting of anti-obesity agents, anti-diabetic mediations, anti-hypertensives, anti-cholesterol agents, and cardiovascular disease treatments. In yet another embodiment, cell therapy is used to treat obesity and/or an obesity associated disorder in the subject comprising isolating autologous adipose-derived stem cells (ASC) from the subject, inducing the ASCs to differentiate into brown adipocytes in vitro, and then implanting the brown adipocytes into the subject.

### DESCRIPTION OF DRAWINGS

FIG. 1A-C presents the expression of polypeptides for MB109 in E. coli cells and purification of the isolated inclusion body. (A) The amino acid sequence of the expressed MB109 polypeptide from E. Coli (SEQ ID NO:2). The first methionine (bold) is engineered for the start of translation. The seven cysteine residues are highlighted in gray. (B) Reduced SDS-PAGE gel of the expressed MB109 polypeptides. Lane 2 and 3 are whole cell lysates before and after IPTG induction. Lane 4 and 5 are supernatant and pellet of the ITPG-induced cell lysate after 16 k×g centrifugation for 10 minutes. Lane 6 is the isolated inclusion body. The black arrow indicates monomeric MB109 polypeptide. (C) Size exclusion chromatographic profiles of Superdex 200 16/60 (left-upper panel) and Superdex 75 16/60 (right-upper panel) loaded with the denatured, monomeric MB109. Bottom panels are reduced SDS-PAGE gel images of each corresponding elution fractions. Gray bars highlight the fractions of monomeric MB109 polypeptide.

FIG. 2A-G provides for the analyses of refolding variables in order to identify optimal refolding conditions for the MB109 polypeptide. (A) Top panel is a non-reduced SDS-PAGE gel image showing the results of refolding in 0-4 M NaCl. Black and gray arrows indicate the refolded functional (bioactive) and chemical (non-bioactive) dimers, respectively. Middle panel shows the details of each refolding condition. Bottom panel is densitometry of the functional dimer bands in the top SDS-PAGE image showing the relative refolding yield in each tested conditions as compared to the highest one (100%). (B-F) Refolding of the MB109 polypeptide at pH 7-10, in 0-4% CHAPS detergent, in different molar ratio of reduced (GSH) and oxidized (GSSG) glutathiones, at 0.05-0.4 mg/mL protein concentration, and a time course of MB109 polypeptide refolding at 4° C. (G) Effect of different additives on the refolding of MB109 polypeptide. The background parameters were 1.5 mM NaCl, pH 8.3, 2% CHAPS, 1/1 mM GSH/GSSG, 0.2 mg/mL protein, 2 mM EDTA and 4° C. for 7 days.

FIG. 3A-C demonstrates the effect of host cell contaminants on the refolding of MB109 polypeptide. Isolated inclusion body, without size exclusion purification, was directly used for refolding at 0.2 μg/mL. Black arrows indicate the refolded functional dimer. (A) Refolding at pH 7-10. The densitometry of the functional band in each condition is shown in black squares and lines. Gray squares and lines are the refolding results from FIG. 2 for comparison. (B and C) Refolding in 0-4 M NaCl and 0-4% CHAPS, respectively.

FIG. 4A-D provides for the purification of refolded bioactive MB109 protein from E. coli. (A) Acidic fractionation by directly titrating the refolded sample with acetic acid. Upper panel is a non-reduced SDS-PAGE image showing the aggregation property of MB109 at pH 5.2, 4.2, 3.2 and 2.2. The Black arrow indicates the functional dimer. S, supernatant; P, pellet. Bottom panel is the corresponding densitometry of the functional dimer in each lane. Black and gray bars represent the relative amounts of functional dimer in supernatants and pellet, respectively. (B) Upper panel, size exclusion chromatographic profile of Superdex 75 and 200 16/60 (connected in series) loaded with acidic fractionated supernatant. The gray bar highlights the pooled fractions for next purification step. Bottom panel is non-reduced SDS-PAGE gel image of each corresponding elution fractions. (C) Ion exchange chromatographic profile of HiTrap SP-FF (1 mL) loaded with the size-exclusion purified sample. Solid line, absorption of 280 nm; dashed line, conductance. Gray bar highlights the pooled fractions containing the functional dimer. (D) Purity analysis of the purified functional dimer on reduced and non-reduced SDS-PAGE gels. The protein was loaded at 0.5, 1, 2 and 10 μg in the absence (left) or presence (right) of 100 mM DTT.

FIG. 5A-B presents cell based activity assays of the purified MB109. (A) Smad1-dependent luciferase reporter assay in mouse myoblast C2C12 cells. Black circles indicate MB109. Gray circles indicate a commercial CHO-derived BMP-9. Black squares indicate E. Coli-derived BMP-2 (S.E.M. are shown, n=3). (B) Cell proliferation assay of AML-12 (left), Hep3B (middle) and HepG2 (right) cells treated with MB109 for 3 days. (S.E.M. are shown, n=3).

FIG. 6A-C shows growth analysis of 15 HCC cells in response to 200 ng/mL of MB109 treatment for 5 days. (A) MB109 is able to inhibit the growth of Hep3B, PLC/PRF/5, SNU-354, SNU-368, SNU-423, SNU-449, SNU-739, SNU-878 and SNU-886. See also FIG. 7. (B) MB109 does not cause growth effect on SNU-182, SNU-398, SNU-475 and SNU-761. See also FIG. 8. (C) MB109 promotes the growth of SNU-387 and HepG2. See also FIG. 9. All cells were grown in media containing 2% FBS, except SNU-368 (10%), SNU-423 (0.5%) and SNU-449 (10%). The representative data of at least three independent experiments is shown. All results are presented as mean±SD, n=4.

FIG. 7A-I shows cell proliferation data of the nine HCC cells that can be inhibited by MB109 treatment. Panels of the left four columns are time-course growth analysis. Cells were cultured in media containing 0.1, 0.5, 2 and 10% FBS. Two hundred ng/mL of MB109 was treated for five days, and the cell numbers were measured every day. Panels of the right column are dosage analysis. Cell numbers were measured after four or five days of ligand treatment. All results are presented as mean±SD, n=3-4.

FIG. 8A-D shows cell proliferation data of the four HCC cells that do not response to MB109 treatment in all tested FBS concentrations. All results are presented as mean±SD, n=3-4.

FIG. 9A-B shows cell proliferation data of the two HCC cells that can be promoted by MB109 treatment. All results are presented as mean±SD, n=3-4.

FIG. 10A-C shows MB109 induces p21 expression, survivin suppression, and G0/G1 cell cycle arrest in Hep3B cells. (A) Expression analyses of p21 by western blot and RT-PCR. (B) RT-PCR analysis of survivin expression. (C) Cell cycle determination by FACScan after PI staining. Left panel shows the raw data of FACScan. Right panel is the numerical conversion of the cell population in G1/0, S, and G2/M phases. The representative data of three independent experiments is shown. RT-PCR results are presented as mean±SD, n=3.

FIG. 11A-D shows ID3 is involved in MB109-induced p21 expression in Hep3B cells. (A) Expression analysis of ID1, ID2, ID3 and ID4 upon 200 ng/mL of MB109 and BMP-2 (control) treatments by RT-PCR. (B) Efficiency of siRNA knock-down of the IDs analyzed by RT-PCR. (C) Expression of p21 mRNA was analyzed in each ID-KD background. Knock-down of ID3 was found to significantly attenuate MB109-induced p21 expression. (***: p<0.0001, one-way ANOVA with Dunnett's post-test) (D) Analysis of MB109-induced ID3 mRNA (left panel) and p21 protein (right panel) expressions in the presence or absence of 50 nM LDN193189, a chemical inhibitor for ALK2/3/6. For all the experiments, MB109 was treated at 200 ng/ml for 24 hours. The representative data of three independent experiments is shown. RT-PCR results are presented as mean±SD, n=3.

FIG. 12A-C shows p38 MAPK controls MB109-induced ID3 and p21 expressions in Hep3B cells. (A) Analysis of MB109-induced ID3 mRNA (left panel) and p21 protein (right panel) expressions in the presence or absence of 50 nM SB202190, a chemical inhibitor for p38 MAPK activity. Cells were exposed to 200 ng/mL of MB109 for 4 hours. (B) Western blot analysis of SMAD1/5/8 and p38 MAPK phosphorylations and ID3 and p21 expressions during 720 minutes of 200 ng/mL of MB109 treatment. (C) A working model of the anti-proliferative BMP-9 signaling pathway in Hep3B cells. The representative results of three independent experiments are shown. RT-PCR results are presented as mean±SD, n=3.

FIG. 13A-F shows that prolonged MB109 treatment reduces different cancer stem cell populations in Hep3B cells. (A) RT-PCR analyses show that the expression levels of p21 (left panel) and ID3 (right panel) were drastically increased during prolonged MB109 treatment (passages #1-9), and were returned to basal levels right after MB109 was removed from the medium (passages #11-21). (B) Expression levels of CD44 (left panel), CD90 (middle panel) and AFP (right panel) were reduced significantly and permanently by the prolonged MB109 treatment. (C) Expression levels of GPC3 (left panel) and ANPEP (right panel) were moderately suppressed by MB109 treatment. (D) Expression levels of CD133 were little or slightly suppressed by MB109 treatment. MB109 was treated at 200 ng/mL. RT-PCR results are presented as mean±SD, n=4. (E and F) Flow cytometry analyses show that the CD44⁺ (left panels) and CD90⁺ (right panels) populations were significantly reduced in the MB109-treated cells at passage #21 (bottom panels) as compared to the untreated Hep3B cells (top panels). Antibodies labeled and unlabeled cells are shown as gray areas and black lines, respectively. A representative data of four independent treatments is shown.

FIG. 14A-G shows that MB109 suppresses Hep3B cell growth and LCSC population in mouse xenograft model. (A) Time course analysis of tumor growth measured three times a week for 30 days. MB109 was injected at 250 (left panel) and 1000 (middle panel) pg/kg intraperitoneally, or 1000 μg/kg intravenously (right panel). Inhibition of tumor growth was observed in all three experimental groups. Red arrows indicate the three time points of injection. The results are shown in means±SD (Sham, n=5; MB109-IP250 and MB109-IP1000, n=6; MB109-IV1000, n=4-5, one mouse was dead at day 8). (B) No difference of the body weight was observed among the mice groups. (C) Tumor weight measured after resection and is shown in scatter plot and means±SD. (**: p<0.005, ***: p<0.0001, Unpaired t test) (D) Visualization of the resected tumors. Scale bars represent 10 mm. (E) Immuno-fluorescence images show that the expression of CD44 on the xenografted tumor tissue of the MB109-1P250 group was reduced as compared to that of the Sham group. CD44 antibodies were conjugated with FITC. Tumor tissues were nuclear counter stained with DAPI. (F and G) Immunohistochemistry images show that the expression of CD90 (F) and AFP (G) on the xenografted tumor tissue of the MB109-IP250 group was reduced as compared to that of the Sham group. Antibody bound regions of CD90 and AFP were visualized with DAB and counter stained with hematoxylin.

FIG. 15A-F provides that MB109 treatment leads to HBx′ cell cycle arrest at G0/1. As a consequence of p21 overexpression and Survivin suppression, MB109 treatment induced cell cycle changes in HBV⁺/HBx⁺ HCC cells: (A) Hep3B, (B) SNU-368, (C) SNU-354, (D) SNU-182, (E) SNU-398, and (F) HepG2. Treatment with MB109 leads to arrest of the cell cycle at the G0/1 phase for most of the cell types.

FIG. 16A-E demonstrates that MB109 is a potent inducer of adipogenesis of brown adipose tissue. Human adipose-derived stem cells (hASCs) were pre-conditioned in the growth medium [G] with various concentrations of ligands for a day, treated in the differentiation medium [D] for 7 days, and subjected to extraction of total RNA. Analysis of expression of aP2 (A), PPARy (B), and C/EBPα (C) genes were carried out using real-time PCR with cyclophilin as an internal control. (D) hASCs were differentiated as described above, incubated with or without 0.5 mM db-cAMP for 6 hours, and then subjected to extraction of total RNA. Results of three independent experiments are presented as means±SD. (E) hASCs grown on cover slip slid chambers were preconditioned in growth medium with vehicle or 100 ng/ml ligands for 2 days, induced differentiation for 10 days, and analyzed using immunocytochemical fluorescence staining of Dapi (blue) and UCP1 (green) (final magnification ×100).

FIG. 17A-E shows that MB109 suppresses weight gain in mice fed a high fat diet by reducing fat mass. (A) Body weight changes of C57BL/6 mice (n=8) fed with a normal chow (NC) diet or 60% Kcal high fat (HF) diet were observed for 9 weeks. Intraperitoneal injection of vehicle (PBS), BMP-2 (100 μg/kg per injection), or MB109 (100 μg/kg per injection) were performed twice a week for 8 weeks. (B) Representative images of epididymal fat tissues are shown. Weights of epididymal fat tissues from the HF/sham and HF/MB109 groups were then analyzed. Results are presented as means±standard deviation (SD). *p<0.05 vs sham control. (C) Representative H&E staining of the subcutaneous and epididymal adipose tissues of animals with average body weights in each group. Scale bars indicate 100 μm (200× magnification). (D) Percentage distribution of cross-sectional areas of adipocytes from each group. (E) Changes in food consumption per mouse for 8 weeks.

FIG. 18A-D demonstrates that MB109 induces browning of the subcutaneous WAT. (A) Real-time PCR was carried out with cyclophilin as an internal control. An average value of UCP1 expression of a HF/sham group (vehicle only) was calculated as 1 for statistical analysis. Distribution of UCP1 expression in each mouse fat tissue sample was displayed as a dot. Results are presented as means±SD. *p<0.05 vs sham control. (B) UCP1 protein expression in subcutaneous WAT and epiren WATs. (C) immunohistochemical staining for UCP-1 in WAT. Representative H&E staining of the subcutaneous adipose tissues. (D) Zoom-in view of the boxed area in (C). Scale bars indicate 100 μm.

FIG. 19A-I provides that MB109 enhances the expression levels of (A) CIDEA and (B) CD137 in the subcutaneous WAT. By contrast, MB109 did not significantly change the expression of (A) CIDEA and (B) CD137 in epiren WAT, or significantly change the expression for (C) Tmem26, (D) Tbx1, (E) Eva 1, (F) Pdk4, (G) resistin or (H) Leptin in subcutaneous WAT. Average values of each gene expression of the HF/sham group were calculated as 1 for statistical analysis. (I) ELISA assay showing no significant change in Leptin expression with MB109 treatment. All results are presented as means±SD. *p<0.05 vs sham control.

FIG. 20A-D shows that intraperitoneal injection of MB109 decreased obesity-associated blood glucose levels. (A) Blood glucose levels of NC and HF groups after 16 hours of fasting are shown. Results are presented as means±SD. (B) Expression levels of GLUT4 in the subcutaneous WAT and epirenal WAT were analyzed using real-time PCR. Expression levels of (C) PEPCK, (D) FAS in the liver were analyzed using real-time PCR. Results are presented as means±SD.

### DETAILED DESCRIPTION

As used herein and in the appended claims, the singular forms “a,” “and,” and “the” include plural referents unless the context clearly dictates otherwise. Thus, for example, reference to “a BMP polypeptide” includes a plurality of BMP polypeptides and reference to “the cancer” includes reference to one or more cancers known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art. Although there are additional methods and reagents similar or equivalent to those described herein, the exemplary methods and materials are presented herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

The term “about” or “approximately” means an acceptable error for a particular value, which depends in part on how the value is measured or determined. In certain embodiments, “about” can mean 1 or more standard deviations. “About”, for example, means that the value differs from a specific value by 1%, 2%, 3%, 4%, 5%, 6%, 7%, 8%, 9%, 10%, 15% or 25% of the recited value.

The term “disorder” as used herein is intended to be generally synonymous, and is used interchangeably with, the terms “disease,” “syndrome,” and “condition” (as in medical condition), in that all reflect an abnormal condition of the body or of one of its parts that impairs normal functioning and is typically manifested by distinguishing signs and symptoms.

The terms “prevent,” “preventing,” and “prevention” refer to a method of delaying or precluding the onset of a disorder; and/or its attendant symptoms, barring a subject from acquiring a disorder, reducing a subject's risk of acquiring a disorder, or inhibiting the progression of the disorder or deterioration of the subject as a result of the disease or disorder.

The term “subject” refers to an animal, including, but not limited to, a primate (e.g., human monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “therapeutically effective amount” refers to the amount of MB109 protein that, when administered, is sufficient to prevent development of, or alleviate to some extent, one or more of the symptoms of a disorder being treated. The term “therapeutically effective amount” also refers to the amount of MB109 protein that is sufficient to elicit the biological or medical response of a cell, tissue, system, animal, or human that is being used by a researcher, veterinarian, medical doctor, or clinician.

The terms “treat,” “treating,” and “treatment” are meant to include alleviating or abrogating a disorder; or one or more of the symptoms associated with the disorder; or alleviating or eradicating the cause(s) of the disorder itself.

The disclosure provides a recombinant polypeptide that can be produced and purified to produce a bioactive protein having BMP-9 activity. The disclosure also provides a simple, straight forward method to produce recombinant TGF-beta superfamily ligands by using a bacterial expression system in combination with protein refolding techniques.

Bone morphogenetic proteins (BMPs) are extracellular growth factors that belong to the transforming growth factor-beta (TGF-beta) superfamily. Among the seventeen members in the BMP subfamily, BMP-9 has been distinguished from other BMPs by its unique receptor-binding specificity and its diverse roles in a variety of cellular processes. For examples, BMP-9 together with BMP-10 signals only through the activin receptor-like kinase 1 (ALK1) to trigger downstream cellular responses, while the other BMP ligands interact promiscuously with different type I receptors. BMP-9 regulates the growth and migration of endothelial cells and therefore plays an essential role in angiogenesis. BMP-9 has been shown to induce apoptosis in prostate cancer cells and to promote the proliferation of ovarian cancer cells. BMP-9 is also one of the most potent BMPs to induce osteogenic differentiation and orthotopic bone formation. BMP-9 promotes chondrogenic differentiation in cultured multipotential mesenchymal cells and articular chondrocytes. Therefore, BMP-9 has been suggested as an effective bone regeneration and tissue repair agent for clinical applications.

BMP-9 is a member of the TGF-beta superfamily, which is responsible for various functions in the human development and adult tissues. BMP-9 directs mesenchymal stem cells to enter into the chondrogenic lineage and induces the proliferation of hematopoietic progenitor cells. BMP-9 is predominantly expressed in the liver of adult animals. At cellular level, the half maximal effective concentration (EC₅₀) of BMP-9 to trigger downstream Smad signaling is around 0.6-1.5 ng/mL. In the human body, BMP-9 circulates in the blood stream at an active concentration of around 2 to 12 ng/ml.

In addition to angiogenesis, osteogenesis and chondrogenesis, BMP-9 is a potent inducer of the cholinergic phenotype in the central nervous system. Therefore, BMP-9 has the potential to be used in regenerative medicine for treating diseases related to cholinergic neurons. In the liver, BMP-9 stimulates cell proliferation of cultured hepatocytes. BMP-9 also inhibits the production of hepatic glucose and activates the expression of several key enzymes in lipid metabolism. Therefore, BMP-9 can be used as a treatment for obesity related disorders and conditions, such as type II diabetes.

Because of the diverse biological functions and pharmaceutical potential, production of recombinant human BMP-9 has garnered significant interest. A mature human BMP-9 contains two identical polypeptides of 110 amino acids. Each of the polypeptides has seven cysteine residues forming three intra-molecular and one inter-molecular disulfide bonds. These seven disulfide bonds form the characteristic cysteine knot and maintain the structural scaffold of an active BMP-9 ligand as a rigid homodimeric protein as revealed by X-ray crystallography. Because of the complex disulfide linking, production of bioactive recombinant human BMP-9 has only been achieved by using mammalian Chinese hamster ovary (CHO) cells. The CHO cell expression system utilizes the natural cellular machinery for folding and making the disulfide bonds. After post-translational modifications, bioactive human BMP-9 is secreted in the culture medium and can be purified by chromatographic methods. The CHO-derived human BMP-9 has a molecular weight close to its theoretic value on SDS-PAGE, indicating that it is not glycosylated.

Although the CHO expression system has an advantage of directly making bioactive recombinant BMP-9 in the cell culture medium, it is costly and time consuming to establish a stable cell line that highly-expresses recombinant BMP-9. These disadvantages provide technical barriers in generating and screening synthetic protein libraries containing tens or hundreds of chimera TGF-beta superfamily ligands. Therefore, the disclosure provides a microbial system adopted to develop a simple, rapid and economical alternative for producing bioactive recombinant polypeptide having BMP-9 activity. When overexpressed in bacteria, such as E. coli, several TGF-beta superfamily proteins aggregate as water-insoluble inclusion bodies which require in vitro denaturation and re-naturation to restore the native protein conformation. Chemical refolding by rapid protein dilution has been developed to produce bioactive recombinant BMP-2 and its Drosophila DPP homolog. Refolding variables and parameters have been discovered to successfully produce other recombinant BMP ligands, such as BMP-3, BMP-6, BMP-2/6 heterodimer, BMP-12 and BMP-13 in high yields. However, the standard refolding conditions are generally not applicable to refold other TGF-beta superfamily ligands.

Provided herein is a polynucleotide having at least 70%, 75%, 80%, 85%, 90%, 91%, 92%, 93%, 94%, 95%, 96%, 97%, 98%, or 99% sequence identity to SEQ ID NO:1 and which encodes a polypeptide comprising a sequence of SEQ ID NO:2. The polynucleotide of SEQ ID NO:1 has been codon optimized for expressing a polypeptide comprising SEQ ID NO:2 in E. coli. It should be noted, however, that any number of additional polynucleotide sequences can be generated that have a certain percent identity to SEQ ID:NO 1 but are instead codon optimized for increasing the expression of the polypeptide of SEQ ID NO:2 in alternate types of bacteria, such as Corynebacterium glutamicum, and Pseudomonas fluorescens. Likewise, any number of mutations can be made to the sequence of SEQ ID NO:1 which still allows for encoding of protein having BMP-9 like activity.

Further provided herein is a polypeptide having at least 85%, 90%, 91%, 92%, 93%, 94%, 95%, 96%, 97%, 98%, or 99% sequence identity to SEQ ID N0:2, wherein the polypeptide retains a methionine at position 1. In a particular embodiment, the disclosure provides for a protein which comprises one or more polypeptides comprising a sequence of SEQ ID NO:2 and/or a percent sequence identity as defined herein to SEQ ID NO:2. SEQ ID NO:2 encodes the mature 110 amino acid region of human BMP-9 (Ser320-Arg429) preceded by Methionine at codon 1 (start codon). It should be noted, however, that any number of mutations, including point mutations, conservative substitutions, deletions and insertions, may be made to sequence of SEQ ID NO:2 that still allows for the generation of a protein which has BMP-9 like activity. In a particular embodiment, the disclosure provides for a bioactive recombinant MB109 protein, which is comprised of a homodimer of two polypeptides having a sequence of SEQ ID NO:2 and/or a percent sequence identity as defined herein to SEQ ID NO:2. In an alternate embodiment, the disclosure provides for a bioactive recombinant MB109 protein, which is comprised of a heterodimer of a polypeptide having a sequence of SEQ ID NO:2 and/or polypeptide(s) having a percent sequence identity as defined herein to SEQ ID NO:2 and which has BMP-9 activity.

“Sequence identity” means that two amino acid sequences are substantially identical (e.g., on an amino acid-by-amino acid basis) over a window of comparison. The term “sequence similarity” refers to similar amino acids that share the same biophysical characteristics. The term “percentage of sequence identity” or “percentage of sequence similarity” is calculated by comparing two optimally aligned sequences over the window of comparison, determining the number of positions at which the identical residues (or similar residues) occur in both polypeptide sequences to yield the number of matched positions, dividing the number of matched positions by the total number of positions in the window of comparison (i.e., the window size), and multiplying the result by 100 to yield the percentage of sequence identity (or percentage of sequence similarity). With regard to polynucleotide sequences, the terms sequence identity and sequence similarity have comparable meaning as described for protein sequences, with the term “percentage of sequence identity” indicating that two polynucleotide sequences are identical (on a nucleotide-by-nucleotide basis) over a window of comparison. As such, a percentage of polynucleotide sequence identity (or percentage of polynucleotide sequence similarity, e.g., for silent substitutions or other substitutions, based upon the analysis algorithm) also can be calculated. Maximum correspondence can be determined by using one of the sequence algorithms described herein (or other algorithms available to those of ordinary skill in the art) or by visual inspection.

As applied to polypeptides, the term substantial identity or substantial similarity means that two peptide sequences, when optimally aligned, such as by the programs BLAST, GAP or BESTFIT using default gap weights or by visual inspection, share sequence identity or sequence similarity. Similarly, as applied in the context of two nucleic acids, the term substantial identity or substantial similarity means that the two nucleic acid sequences, when optimally aligned, such as by the programs BLAST, GAP or BESTFIT using default gap weights (described elsewhere herein) or by visual inspection, share sequence identity or sequence similarity.

One example of an algorithm that is suitable for determining percent sequence identity or sequence similarity is the FASTA algorithm, which is described in Pearson, W. R. & Lipman, D. J., (1988) Proc. Natl. Acad. Sci. USA 85:2444. See also, W. R. Pearson, (1996) Methods Enzymology 266:227-258. Preferred parameters used in a FASTA alignment of DNA sequences to calculate percent identity or percent similarity are optimized, BL50 Matrix 15: −5, k-tuple=2; joining penalty=40, optimization=28; gap penalty −12, gap length penalty=−2; and width=16.

Another example of a useful algorithm is PILEUP. PILEUP creates a multiple sequence alignment from a group of related sequences using progressive, pairwise alignments to show relationship and percent sequence identity or percent sequence similarity. It also plots a tree or dendogram showing the clustering relationships used to create the alignment. PILEUP uses a simplification of the progressive alignment method of Feng & Doolittle, (1987) J. Mol. Evol. 35:351-360. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using the following parameters: default gap weight (3.00), default gap length weight (0.10), and weighted end gaps. PILEUP can be obtained from the GCG sequence analysis software package, e.g., version 7.0 (Devereaux et al., (1984) Nuc. Acids Res. 12:387-395).

Another example of an algorithm that is suitable for multiple DNA and amino acid sequence alignments is the CLUSTALW program (Thompson, J. D. et al., (1994) Nuc. Acids Res. 22:4673-4680). CLUSTALW performs multiple pairwise comparisons between groups of sequences and assembles them into a multiple alignment based on sequence identity. Gap open and Gap extension penalties were 10 and 0.05 respectively. For amino acid alignments, the BLOSUM algorithm can be used as a protein weight matrix (Henikoff and Henikoff, (1992) Proc. Natl. Acad. Sci. USA 89:10915-10919).

“Functional” or “activity” refers to a polypeptide which possesses either the native biological activity of the naturally-produced proteins of its type, or any specific desired activity, for example as judged by its ability to bind to receptors or affect cell cycle regulation. Thus, for example, an MB109 polypeptide having BMP-9 activity refers to a MB109 polypeptide that can form intra- and inter-disulfide bonds similar to BMP-9. An MB109 protein refers to a dimerized MB109 polypeptide that has BMP-9 activity (e.g., any number of the biological activities described above for BMP-9).

In order to produce bioactive MB109 protein from bacteria in high yield, a comprehensive analysis of the rapid dilution refolding method was performed. After important variables were identified, the variables were optimized. A simple purification scheme was also developed to purify bioactive MB109 protein from bacteria for receptor binding analysis, Smad1-dependent luciferase reporter assays and cell proliferation assays.

The disclosure provides a simple, straight forward method to produce recombinant TGF-beta superfamily ligands by using a bacterial expression system in combination with protein refolding techniques. Examples of bacteria which can be used to produce the MB109 polypeptide of the disclosure include, but are not limited to, Escherichia coli, Corynebacterium glutamicum, and Pseudomonas fluorescens. In a particular embodiment, the MB109 polypeptide are produced and isolated from E. coli. The production methods disclosed herein take advantage of molecular tools in combination with the robust protein expression capability of bacteria, such as E. coli, to produce bioactive recombinant MB109 in high yield. The production methods disclosed herein are particularly suitable in generating a protein library of tens or hundreds of synthetic TGF-beta based chimera. Further, the methods of the disclosure can be incorporated into a mid or high throughput screening systems in order to identify novel biologics in a manner similar to antibody discovery.

One of the main limitations in expressing TGF-beta family members from prokaryotic cells is protein refolding. To overcome this bottlenecking step, a comprehensive study of variables for a rapid dilution refolding method was examined. It was found that the refolding of MB109 protein is a very slow process, which takes more than 7 days to reach stationary at 4° C. The refolding efficiency is dependent on the NaCl, CHAPS and protein concentrations in the refolding solution. Moreover, the refolding efficiency was sensitive to the buffer pH and to the presence of several commonly used denaturants and aggregation suppressors. These variables therefore provide criteria for bioactive MB109 protein folding from a bacterial inclusion body. Redox conditions did not appear to have an effect on folding. So long as the amount of reducing agent (e.g., glutathione (GSH)) equals or exceeds that of an oxidizing agent (e.g., glutathione disulfide (GSSG)), bioactive MB109 protein can be refolded with the same efficiency. In a particular embodiment, the disclosure provides for MB109 protein folding conditions which include one or more of the following: a buffer pH between about 7.0 and 9; a detergent concentration between about 1 and 5%; a salt concentration between about 0.5 M and 3 M; a redox system having a molar ratio between about 10:1 and 1:10 of a reductant to oxidant; a protein concentration of about 0.4 mg/mL or lower; and a refolding temperature/duration of about 4° C. for about 7 or more days. In a further embodiment, the disclosure provides for MB109 protein folding conditions comprising: a buffer pH between about 8.0 and 8.5; a CHAPS concentration between about 2 and 4%; an NaCl concentration between about 1 and 2M; a redox system having a GSH/GSSG molar ratio between about 2:1 and 1:1; a protein concentration of about 0.1 mg/mL or lower; and a refolding temperature/duration of about 4° C. for about 9 days.

It was further determined that the presence of bacterial host cell contaminants had little to no detrimental effect on the folding of an MB109 protein of the disclosure. The results indicate that washed inclusion bodies containing MB109 polypeptide of the disclosure can be solubilized directly and used for refolding without any sacrifice to the refolding efficiency. This property is particularly useful when a large number of targets need to be refolded in a high throughput manner or when the MB109 protein needs to be produced in large yields.

While recombinant activin A, a member of TGF-beta superfamily ligands, can be refolded to varying degrees with different detergents, TDCA (sodium taurodeoxycholate) was found to be the most effective. The detergent-like CHES (2-(cyclohexylamino)ethanesulfonic acid) has also been used to refold recombinant BMP-2 with high yield in addition to CHAPS (3-[(3-cholamidopropyl) dimethylammonio]-1-propanesulfonate). The use of different detergents to refold activin A and BMPs indicates that detergents are a useful additive for refolding TGF-beta superfamily ligands. Accordingly, in a particular embodiment, a method to refold the MB109 protein of the disclosure utilizes one or more detergents including, but not limited to, Triton X-100, Triton X-114, NP-40, BRij-35, Brij-58, Tween 20, Tween 80, octyl glucoside, octyl thioglucoside, sodium dodecyl sulfate (SDS), TDCA (sodium taurodeoxycholate), CHES, CHAPS, and CHAPSO (3-([3-cholamidopropyl] dimethylammonio)-2-hydroxy-1-propanesulfonate).

In the experiments presented herein, it was shown that the MB109 protein of the disclosure had similar bioactivity as CHO-derived human BMP-9 in terms of signaling capability. Further experiments demonstrated that the MB109 protein of the disclosure bound specifically to ALK1, ActRIIb and BMPRII receptors, which is consistent with the data previously studied with CHO-derived human BMP-9. Moreover, the MB109 protein of the disclosure induces opposite growth effects on different liver cell lines in cell proliferation assays. Further studies demonstrate the anti-proliferative effect of the MB109 protein disclosed herein on mouse hepatocyte AML-12 cells and human hepatoma Hep3B cells. Since the half maximal effective concentration of these growth effects are in the range of the circulating BMP-9 levels in adult mouse and human plasma (2-12 ng/mL), the MB109 protein disclosed herein could be used to treat liver cancer and other similar types of cancers.

The liver can be affected by primary liver cancer, which arises in the liver, or by cancer which forms in other sites which then spreads to the liver. Most liver cancer is secondary or metastatic, meaning it started elsewhere in the body. Primary liver cancer, which starts in the liver, accounts for about 2% of cancers in the U.S., but up to half of all cancers in some undeveloped countries. This is mainly due to infections by contagious viruses, such as Hepatitis B virus, that predisposes a person to liver cancer. Almost a third of the world's population has been infected with Hepatitis B. Those who develop liver cancer only have a 5 year survival rate of about 10%. A strong correlation has been shown between infant Hepatitis B vaccination and a reduction in the incidence of liver cancer. Epidemiologically, it would take decades to eliminate Hepatitis B, like smallpox, and until then, the prevalence of hepatocellular carcinomas (HCC) is expected to remain high.

HCC is the fifth most common cancer and accounts for the third leading cancer deaths in the world. HCC life expectancy is poor due to 40% of patients being diagnosed at an advanced stage of cancer. Hepatitis B (HBV) virus infection is considered to be the leading cause of HCCs. 350 million people are chronically infected with HBV, and of which 25% further develop HCC. The main treatment for HCC is surgical resection, chemotherapy, and radiofrequency ablation. Very few medications are available to slow down the progression of the HCC. One such medication is Sorafenib, a small molecule inhibitor for MAP-Kinase pathway. Due to the cost of Sorafenib, its use is limited, especially in underdeveloped countries. There are currently no drugs approved to specifically target HBV induced HCC.

HCC, like many other cancers, develop multiple signaling mutations in order to override regulation and apoptosis. Although there are many known mutations in HCC, one of the most characterized is a mutation to transforming growth factor-beta (TGF-beta) pathway, including SMAD2 and SMAD4 gene mutations and reduced TGF-beta type II receptor expression. The mutation to SMAD4 is especially important, since SMAD4 is utilized by most members of the TGF-beta superfamily. Mutation to SMAD4 can lead to the shutting down of multiple TGF-beta related signaling pathways. Furthermore, in vivo transgenic mouse models utilizing dominant-negative TGF-beta type II receptor mutations, have demonstrated in hepatocytes, increased incidence, size and multiplicity of chemically induced tumor formation. The importance of TGF-beta signaling in regulating the initiation and proliferation of HCC is therefore well recognized in the art.

In the experiments presented herein, it was found that the MB109 protein disclosed herein played an important role in suppressing the proliferation of liver cancer characterized by an HBV infection, especially those liver cancers which produce HBx. It was further found, that cell cycle regulation can be restored by treating with the MB109 protein disclosed herein. In particular, the expression of p21 and the suppression of Survivin can be restored in cells which have a disrupted p53 pathway (e.g., HBx). p53 is the most frequently altered gene in cancers. However, treatment with the MB109 protein disclosed herein did not revive the p53 pathway itself, but utilized an alternative pathway. This alternative pathway is most likely a SMAD pathway as there is an established relationship between SMAD, p21 and BMP, and further between the SMAD3 pathway and HBx in HCCs. As the MB109 protein of the disclosure can restore cell regulation in cells with a disrupted p53 pathway, it can be expected that the MB109 protein of the disclosure can be used to treat most if not all cancers and tumors. Examples of cancers and tumors in which the MB109 protein disclosed herein can treat would include, but are not limited to, adrenocortical carcinoma; anal cancer; bladder cancer; brain tumors, including gliomas, cerebellar astrocytomas, cerebral astrocytomas, and ependymomas; breast cancer; gastrointestinal carcinoid tumors; cervical cancer; colon cancer; endometrial cancer; esophageal cancer; extrahepatic bile duct cancer; ewings family of tumors (PNET); extracranial germ cell tumors; extragonadal germ cell tumors; eye cancer, including intraocular melanomas; gallbladder cancer; gastric cancer (stomach); gestational trophoblastic tumor; head and neck cancer; hypopharyngeal cancer; islet cell carcinoma; kidney Cancer (renal cell cancer); laryngeal cancer; leukemias, including acute lymphoblastic leukemia, acute myeloid leukemia, acute myeloid leukemia, chronic lymphocytic leukemia, chronic myelogenous leukemia, and hairy cell leukemia; lip and oral cavity cancer; liver cancer; lung cancer; lymphomas, including AIDS-Related lymphomas, central nervous system (Primary) lymphomas, cutaneous T-Cell lymphomas, Hodgkin's Disease, and Non-Hodgkin's Disease; malignant mesothelioma; melanoma; merkel cell carcinoma; metasatic squamous neck cancer with occult primary; multiple myeloma and other plasma cell neoplasms; mycosis fungoides; myelodysplastic syndrome; myeloproliferative disorders; nasopharyngeal cancer; neuroblastoma; oral cancer; oropharyngeal cancer; osteosarcoma; ovarian epithelial cancer; ovarian germ cell tumor; pancreatic cancers, including exocrine and islet cell carcinomas; paranasal sinus and nasal cavity cancer; parathyroid cancer; penile cancer; pituitary cancer; plasma cell neoplasm; prostate cancer; rhabdomyosarcoma; rectal cancer; renal cell cancer (cancer of the kidney); transitional cell renal pelvis and ureter cancer; salivary gland cancer; sezary syndrome; skin cancers, including cutaneous T-Cell lymphomas, Kaposi's sarcomas, and melanomas; small intestine cancer; soft tissue sarcoma; stomach cancer; testicular cancer; malignant thyoma; thyroid cancer; urethral cancer; uterine cancer; vaginal cancer; vulvar cancer; and Wilms' tumor. In a further embodiment, the MB109 protein disclosed herein can be used to treat liver cancer in a subject. In a further embodiment, MB109 protein disclosed herein can be used to treat any disease, disorder or condition in a subject which can be ameliorated by restoring cell cycle regulation that has been disrupted.

The disclosure also demonstrates that MB109 protein can be used to treat obesity and obesity-related disease and disorders. An imbalance of energy intake to energy expenditure causes storage of excess energy as triglycerides in adipose tissues, resulting in obesity. Prevalence of obesity and obesity-related type 2 diabetes has become a major economic and medical burden worldwide. While white adipose tissues (WATs) function as a storage depot of lipids, brown adipose tissues (BATs) dissipate energy as heat by thermogenesis. WAT, the major type of adipose tissues, is also an endocrine organ secreting adipokines which plays a key role in pathogenesis of obesity and type 2 diabetes. Dysfunctional secretion of pro- and anti-inflammatory adipokines, resulting from obesity, has been associated with insulin resistance, a hallmark of type 2 diabetes.

BAT, whose activity has been reported to be inversely related with age and obesity, is distributed primarily at the neck region. Short-term exposure to cold temperature stimulates the β3-adrenoreceptor on the plasma membrane of brown adipocytes and consequently activates breakdown of triglycerides stored in brown adipocytes. With high mitochondrial content, brown adipocytes utilize uncoupling protein 1 (UCP1) in the inner membrane of mitochondria to dissipate chemical energy into heat. Unlike WAT, BAT is highly vascularized and derived from myf5 muscle lineage cells. In addition to thermogenesis, BAT has been reported to improve glucose homeostasis and insulin sensitivity. Recent studies identified beige/brite adipocytes or recruitable brown adipocytes in the WAT with brown adipocyte-like function. Analysis of gene expression of adult human BAT displayed expression patterns of genes distinct to beige adipocytes.

The bone morphogenetic protein (BMP) signaling pathway has been reported to play a key role in adipogenesis and osteogenesis. While most BMPs interact promiscuously with type I receptors, BMP-9 and -10 bind with high affinity to the activin receptor like kinase 1 (ALK1). Studies indicate that BMP-2, -4, -6, -7, and -9 promote adipogenesis of mesenchymal stem cells (MSCs). Although BMP-2, -4, and -6 could not induce expression of UCP-1, a brown adipocyte marker gene, BMP-7 promotes brown adipogenesis with marked induction of UCP1 and mitochondrial biogenesis. In addition to BMP-7, BMP-8b has also been reported to enhance thermogenesis of BAT with central and peripheral actions. It has been demonstrated that mice deficient of BMP type IA receptor in Myf5-lineage cells displayed a much smaller BAT size and induced UCP1 gene expression in WATs, indicating the existence of a compensatory mechanism to regulate thermogenesis by browning of white adipocytes.

BMP-9, whose expression is highest in liver cells, has been demonstrated to regulate expression of enzymes involved in glucose homeostasis. Administering insulin results in an acute reduction in blood glucose levels that dissipates by 2 hours. By contrast, a bolus injection of CHO-derived BMP-9 reduced blood glucose levels in diabetic mice with maximal reduction around 30 hours after injection. In addition, administration of anti-BMP-9 antibody to fasted rats induced glucose intolerance and insulin resistance. These results indicate that BMP-9 plays a prominent role in glucose homeostasis.

In the experiments presented herein, it was observed that the MB109 protein of the disclosure enhanced brown adipogenesis of human adipose tissue derived stem cells (hASCs). On this basis, MB109 protein can be used to improve glucose metabolism by regulating expression of brown adipogenic genes. Systemic injection of the MB109 protein intraperitoneally (200 μg/kg/week) suppressed weight gain in an obese mouse model and decreased 16 hour fasting blood glucose levels. Brown adipogenic gene expression was observed in the subcutaneous WAT but not in the visceral fat tissues from MB109 injected mice, indicating that MB109 protein of the disclosure induce browning of subcutaneous WAT thereby counteracting the adverse effects resulting from high fat diet-induced obesity. In addition, systematic injection of the MB109 protein enhanced fatty acid synthase (FAS) expression in the liver of obese mice, which may help attenuate obesity-associated increase of blood glucose levels. Periodical injection of the MB109 protein reduced the size of the white adipose tissue from high fat diet fed mice, by counteracting weight gain. Treatment with the MB109 protein displayed preferential browning at the subcutaneous WAT with enhanced gene expression of UCP1 and Cidea as well as a selective beige marker gene such as CD137. In addition to its role as a beige cell marker gene, CD137 has been reported to function in immune cells and regulate glucose metabolism. An animal study demonstrated that stimulation of CD137 using agonistic antibody reduced body weight in high fat diet-mediated obesity mice and improved glucose intolerance. Visceral WATs have been reported to be closely associated with development of obesity-related metabolic diseases, while the subcutaneous WAT is not. Preferential browning at the subcutaneous WAT by periodical injection of the MB109 protein may explain the protective roles of the subcutaneous WAT against obesity-related metabolic diseases.

While mice deficient of BMPR1A in the muscle-lineage cells displayed severe reduction of the BAT and browning at both subcutaneous and epididymis WATs, periodical injection with the MB109 protein of the disclosure did not change the size of BAT or change the expression levels of UCP1 in the BAT. In addition, the MB109 protein disclosed herein induced browning at the subcutaneous WAT but not at the epirenal or epididymis WATs. Although the MB109 protein induced browning of the subcutaneous WAT, it induced only a few selective beige marker genes such as CD137. Therefore, the degree to induce browning WATs may be determined by capacity of the BAT. In addition, it is plausible to hypothesize that the BAT alone may not be enough to counteract pathophysiology resulting from obesity or that browning WATs may have specialized roles in counteracting pathophysiology of obesity. While mice in the study were not exposed to low temperatures, they did not show any changes in the core body temperature. Thus, browning at the subcutaneous WAT by the MB109 protein does not appear to modulate temperature, but rather is useful for counteracting pathophysiology associated with high fat diet-induced obesity. The capacity of the MB109 protein to induce brown adipogenesis was also confirmed using hASC. The disclosure provides in the first instance that MB109 regulates brown adipogenesis and browning of WATs.

BMP-7 and BMP-8b among BMP family ligands have been reported to induce brown adipogenesis. Embryos deficient in BMP-7 displayed marked reduction of BAT sizes and near complete absence of UCP1 expression. Further, BMP-7 treatment induced the commitment of mesenchymal progenitor cells to brown adipocytes. Expression levels of BMP-8b in the BAT are much higher than that in the subcutaneous or gonadal WAT. Although no description of browning of WATs in mice deficient of BMP-8b has been reported, a possible role of BMP-8b in browning of WATs cannot be ruled out. Moreover, BMP-8b plays a central role in the regulation of thermogenesis by being highly expressed in the hypothalamus. Since BMP-7, BMP-8b, and BMP-9 display distinct tissue distribution patterns during development, their capacity to induce brown adipogenesis may be distinct or complementary to each other in response to various physiological conditions.

Receptors for BMP family ligands are composed of type I and type II receptors. BMPR-II functions as a type II receptor for BMP-7, BMP-8b, and BMP-9. While BMPR-IA and BMPR-IB are the type I receptor for BMP-7 and BMP-8b, ALK-I receptor is the only type I receptor for BMP-9. Deficiency of BMPR-IA or ACVRI, but not BMPR-IB, impairs development of constitutive BAT, suggesting that TGF-beta superfamily ligands other than BMP-7, -8b, and -9 may also play a role in brown adipogenesis.

A bolus injection of CHO-derived human BMP-9 (5 mg/kg) decreased blood glucose levels with a maximum effect at 30 hours after injection. In order to rule out any acute effects of MB109 protein on glucose metabolism, blood glucose levels were measured 5 days after the injection of ligands. Mechanisms underlying the improved obesity-mediated glucose metabolism by periodical injection of low dose MB109 protein (200 μg/kg/week) of the disclosure may be different from that of a bolus injection of high dose CHO-derived human BMP-9 (5 mg/kg). Indeed, while bolus injection of high dose CHO-derived human BMP-9 suppressed PEPCK expression in the liver, periodical injection of low dose MB109 protein did not suppress PEPCK mRNA expression in the liver. Nevertheless, both periodical injection of low dose MB109 protein of the disclosure and bolus injection of high dose CHO-derived human BMP-9, enhanced fatty acid synthase mRNA expression in the liver, which may help convert glucose in the liver into fatty acid and lower blood glucose levels. In comparison to the studies presented herein, there have been no previous reports that a bolus injection of high dose CHO-derived human BMP-9 decreases the mass of WATs or induces the browning of WATs.

Accordingly, the studies presented herein, demonstrate the first instance of reducing fat mass and obesity-associated dysregulated blood glucose levels in HF diet-induced obese mice via inducing browning of the subcutaneous WAT and up-regulating FAS mRNA expression in the liver, by systemically administering MB109 protein. Also presented herein, is the first showing that hASC can be differentiated into brown adipocytes by treating with proteins that have BMP-9 like activity.

The disclosure therefore provides for MB109 protein that can be used to treat obesity and obesity associated disorders, including, but not limited to, type 2 diabetes; cancers such as endometrial, breast and colon cancer; heart disease; stroke; hypertension; liver disease; gallbladder disease; osteoarthritis; metabolic syndrome; polycystic ovary syndrome (PCOS); reproductive hormonal abnormalities; and dyslipidemia. The disclosure also provides that the MB109 protein can be used in any disease, disorder or condition that can be ameliorated by activating brown adipogenesis and/or suppressing adverse effects due to high fat diet-induced obesity. In addition to treating obesity or obesity related disorders, the MB109 protein of the disclosure can also be used for cell therapy, by inducing autologous ASC to differentiate into brown adipocytes in vitro, which can then be implanted back into obese patients.

The disclosure provides for pharmaceutical compositions comprising MB109 protein, as an active ingredient in a pharmaceutically acceptable vehicle, carrier, diluent, or excipient, or a mixture thereof, and/or in combination with one or more pharmaceutically acceptable excipients or carriers.

The terms “active ingredient” and “active substance” refer to MB109 protein alone or with additional therapeutic agents (e.g., chemotherapeutic agents), which is/are administered, alone or in combination with one or more pharmaceutically acceptable excipients or carriers, to a subject for treating, preventing, or ameliorating one or more symptoms of a disease, disorder, syndrome or condition.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function do not include modifying the duration or place of release of the active substance from a dosage form as compared with a conventional immediate release dosage form.

Disclosed herein also are pharmaceutical compositions in a dosage form for parenteral administration to a subject, which comprise a MB109 protein, and one or more pharmaceutically acceptable excipients or carriers.

The disclosure further provides pharmaceutical compositions in modified release dosage forms, which comprise a MB109 protein as disclosed herein, and one or more release controlling excipients or carriers as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof The pharmaceutical compositions may also comprise non-release controlling excipients or carriers.

The disclosure also provides for pharmaceutical compositions in enteric coated dosage forms, which comprise a MB109 protein of the disclosure, and one or more release controlling excipients or carriers for use in an enteric coated dosage form. The pharmaceutical compositions may also comprise non-release controlling excipients or carriers.

Additionally disclosed herein are pharmaceutical compositions in a dosage form that has an instant releasing component and at least one delayed releasing component, and is capable of giving a discontinuous release of a MB109 protein disclosed herein in the form of at least two consecutive pulses separated in time from 0.1 up to 24 hours. The pharmaceutical compositions comprise a MB109 protein, and one or more release controlling and non-release controlling excipients or carriers, such as those excipients or carriers suitable for a disruptable semi-permeable membrane and as swellable substances.

Disclosed herein are pharmaceutical compositions that comprise about 0.1 to about 1000 mg, about 1 to about 500 mg, about 2 to about 100 mg, about 1 mg, about 2 mg, about 3 mg, about 5 mg, about 10 mg, about 20 mg, about 25 mg, about 30 mg, about 40 mg, about 50 mg, about 100 mg, about 500 mg of MB109 protein in the form of sterile lyophilized, or partially broken cake for parenteral administration. The pharmaceutical compositions may further comprise stabilizers, such as mannitol.

Disclosed herein are pharmaceutical compositions that comprise about 0.1 to about 1000 mg, about 1 to about 500 mg, about 2 to about 100 mg, about 1 mg, about 2 mg, about 3 mg, about 5 mg, about 10 mg, about 20 mg, about 25 mg, about 30 mg, about 40 mg, about 50 mg, about 100 mg, about 500 mg of MB109 protein in the form of enteric coated tablets for oral administration. The pharmaceutical compositions further comprise the inactive ingredients, such as acacia, FD&C Blue No. 1, D&C Yellow No. 10 Aluminum Lake, lactose, magnesium stearate, starch, stearic acid and talc.

The pharmaceutical compositions disclosed herein may be disclosed in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient (s) sufficient to produce the desired therapeutic effect, in association with the required pharmaceutical carriers or excipients. Examples of unit-dosage forms include ampoules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The MB109 protein of the disclosure may be administered alone, or in combination with one or more other therapeutic agents disclosed herein, and/or with one or more other active ingredients. For example, the MB109 protein of the disclosure may be administered with chemotherapeutic agents or anti-obesity agents. The pharmaceutical compositions that comprise a MB109 protein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The pharmaceutical compositions disclosed herein may be administered at one, or multiple intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the administration of a MB109 protein may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of a MB109 protein disclosed herein may be given continuously or temporarily suspended for a certain length of time (i.e., a “drug holiday”).

Once improvement of the patient's conditions has occurred, a maintenance dose is administered, if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The pharmaceutical compositions disclosed herein may be disclosed in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

The pharmaceutical compositions disclosed herein may be disclosed as enteric-coating tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates.

The pharmaceutical compositions disclosed herein for oral administration may be also disclosed in the forms of liposomes, micelles, microspheres, or nanosystems. Micellar dosage forms can be prepared as described in U.S. Pat. No. 6,350,458.

The pharmaceutical compositions disclosed herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions disclosed herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In a particular embodiment, the pharmaceutical compositions are disclosed as ready-to-use sterile solutions. In another embodiment, the pharmaceutical compositions are disclosed as sterile dry soluble products, including lyophilized powders and hypodermic tablets, to be reconstituted with a vehicle prior to use. In yet another embodiment, the pharmaceutical compositions are disclosed as ready-to-use sterile suspensions. In yet another embodiment, the pharmaceutical compositions are disclosed as sterile dry insoluble products to be reconstituted with a vehicle prior to use. In still another embodiment, the pharmaceutical compositions are disclosed as ready-to-use sterile emulsions.

The pharmaceutical compositions disclosed herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In a certain embodiment, the pharmaceutical compositions disclosed herein are dispersed in a solid inner matrix, which is surrounded by an outer polymeric membrane that is insoluble in body fluids but allows the active ingredient in the pharmaceutical compositions diffuse through. Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate. Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

The pharmaceutical compositions disclosed herein may be administered topically to the skin, orifices, or mucosa. The topical administration, as used herein, include (intra)dermal, conjunctival, intracorneal, intraocular, ophthalmic, auricular, transdermal, nasal, vaginal, uretheral, respiratory, and rectal administration.

The pharmaceutical compositions disclosed herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions disclosed herein may also comprise liposomes, micelles, microspheres, nanosystems, and mixtures thereof.

Pharmaceutically acceptable carriers and excipients suitable for use in the topical formulations disclosed herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

The pharmaceutical compositions may also be administered topically by electroporation, iontophoresis, phonophoresis, sonophoresis and microneedle or needle-free injection, such as POWDERJECT® (Chiron Corp., Emeryville, Calif.), and BIOJECT® (Bioject Medical Technologies Inc., Tualatin, Oreg.).

The pharmaceutical compositions disclosed herein may be disclosed in the forms of ointments, creams, and gels. The pharmaceutical compositions disclosed herein may be administered intranasally or by inhalation to the respiratory tract. The pharmaceutical compositions may be disclosed in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be disclosed as a dry powder for insufflation, alone or in combination with an inert carrier such as lactose or phospholipids; and nasal drops. For intranasal use, the powder may comprise a bioadhesive agent, including chitosan or cyclodextrin.

The pharmaceutical compositions disclosed herein may be formulated as a modified release dosage form. As used herein, the term “modified release” refers to a dosage form in which the rate or place of release of the active ingredient (s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient (s) can also be modified by varying the particle sizes and polymorphorism of the active ingredient(s).

The MB109 protein disclosed herein may also be combined or used in combination with other agents useful in the treatment, prevention, or amelioration of one or more symptoms of a neoplasia-mediated disorder (e.g., cancer) and/or obesity associated disorder (e.g., type II diabetes) the therapeutic effectiveness of MB109 protein described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a MB109 protein as disclosed herein. When a MB109 protein as disclosed herein is used contemporaneously with one or more other drugs, a pharmaceutical composition containing such other drugs in addition to the MB109 protein as disclosed herein may be utilized, but is not required. Accordingly, the pharmaceutical compositions disclosed herein include those that also contain one or more other active ingredients or therapeutic agents, in addition to the MB109 protein as disclosed herein.

In certain embodiments, a MB109 protein as disclosed herein can be combined with one or more chemotherapeutic agents, including, but not limited to, cancer immunotherapy monoclonal antibodies, alkylating agents, anti-metabolites, mitotic inhibitors, anti-tumor antibiotic agents, topoisomerase inhibitors, photosensitizers, tyrosine kinase inhibitors, and anti-cancer agents.

In additional embodiments, a MB109 protein as disclosed herein can be combined with one or more anti-obesity agents including orlistat, lorcaserin, sibutramine, rimonabant, metformin, exenatide, pramlintide, and a phentermine/topiramate; anti-diabetic mediations including insulin, glyburide, glimepiride, glipizide, acarbose, miglitol, voglibose, pioglitazone, and rosiglitazone; antihypertensives including diuretics, vasodilators, peripheral adrenergic inhibitors, calcium channel blockers, angiotensin II receptor blockers, alpha-2 receptor agonists, and central agonists; anti-cholesterol agents including statins, cholestyramine, gemfibrozil, and pantethine; and cardiovascular disease treatments, including diuretics, angiotensin-converting enzyme (ACE) inhibitors or beta blockers, and blood thinning medications.

In alternate embodiments, a MB109 protein as disclosed herein can be administered in combination with other classes of therapeutic agents, including, but not limited to, endothelin converting enzyme (ECE) inhibitors, such as phosphoramidon; thromboxane receptor antagonists, such as ifetroban; potassium channel openers; thrombin inhibitors, such as hirudin; fibroblast growth factors; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y (AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; H MG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-adrenergic agents; beta-adrenergic agents, such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

For use in the therapeutic applications as described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise a MB109 protein as described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a MB109 protein as described herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a MB109 protein as described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The following examples are intended to illustrate but not limit the disclosure. While they are typical of those that might be used, other procedures known to those skilled in the art may alternatively be used.

### Examples

### Molecular Biology and E. coli Cell Culture

The gene of mature human BMP-9 (Ser320-Arg429), containing a 5′ start codon and a 3′ stop codon, was synthesized by Genscript (NJ, USA). The codon usage was optimized for maximal E. coli expression. The synthetic gene was ligated into a pET21a vector (Novagen, USA) between the NdeI and XhoI restriction enzyme sites to make the expression plasmid. The sequence was confirmed by DNA sequencing after cloning.

For MB109 polypeptide expression, the plasmid was transformed into BL21 E. coli by heat shock. The transformants were selected on an LB-broth agar plate, containing 100 μg/mL ampicillin (LBamp). A single colony was inoculated in LBamp medium (10 mL) and incubated aerobically at 37° C. for 16 hours. The overnight culture was diluted 200-fold into fresh LBamp medium (1 L) and incubated aerobically in a shaking incubator at 37° C. When the cell density reached OD₆₀₀ ˜0.4, 0.5 mM IPTG (isopropyl β-D-1-thiogalactopyranoside) was added to induce the protein expression. After 20 hours of induction at 37° C., the cells were harvested by centrifugation at 8,000×g for 20 minutes.

Inclusion Body Isolation and Chromatographic Purification of Denatured MB109.

To isolate the inclusion body, the cells was re-suspended in deionized water in the presence of 1 mM phenylmethylsulfonyl fluoride (PMSF) and lysed by passing through a Nano DeBEE microfluidizer three times. The inclusion bodies in the cell lysate were precipitated by centrifugation at 12,000×g for 20 minutes, and washed three times by re-suspension in deionized water and centrifugation at 12,000×g for 20 minutes. The washed inclusion body was dissolved in E2 Buffer [50 mM Tris-HCl (pH 8.2), 8 M urea, 40 mM DTT and 2 mM EDTA] by sonication at 4° C. The denatured protein solution was filtered through a 0.2 μm membrane and stored at −80° C. Gel filtration chromatography was performed with an Akta Prime plus FPLC using E Buffer [50 mM Tris-HCl (pH 8.2), 6 M urea, 250 mM NaCl and 1.0 mM DTT] as the elution solution. The fractions containing monomeric MB109 polypeptide were pooled, concentrated to 20 mg/mL by Vivaspin20 (10,000 MWCO) (Sartorius stedim biotech) concentrators and stored at −80° C. The protein concentration was determined by using the Bio-Rad Protein Assay kit (Bio-Rad Laboratories, USA).

Chemical Refolding and Purification of Refolded MB109.

Small-scale refolding screening analyses were performed with a final volume of 1-5 mL in either 1.5-mL Eppendorf tubes or 14-mL conical tubes. The pH of the refolding solutions was titrated at 4° C. For rapid dilution, the purified monomeric MB109 was diluted directly into cold refolding solutions, mixed rapidly by vortexing and incubated at 4° C. To visualize the progress of refolding, sample solutions were centrifuged at 16 k×g for 10 minutes to pellet visible aggregates. The supernatants were diluted 5-fold in a U2-4.5-NS buffer solution (1% acetate, pH 4.5, and 8M urea) and concentrated 25-fold by vivaspin6 (10,000 MWCO) concentrators. The concentrated protein samples were run on non-reduced 12% SDS-PAGE electrophoresis and stained by Coomassie-based InstantBlue (Expedeon, UK). The densitometry of the functional dimer bands on the SDS-PAGE gel images was analyzed by using the GeneTools image analysis software (Synoptics, UK).

To scale-up refolding for purifying the refolded MB109 protein, 100 mg of purified monomeric MB109 polypeptides were diluted in refolding solution (1 L), containing 50 mM Tris-HCl (pH 8.3), 1.5 M NaCl, 3% CHAPS, 2:1 mM GSH/GSSG and 2 mM EDTA, and incubated at 4° C. for 9 days. For acidic fractionation, the sample solution was titrated to pH 3.2 using acetic acid and incubated at room temperature (24° C.) for 16 hours. The aggregates were removed by 0.2 μm filtration and the filtrate was concentrated by a vivaflow200 (10,000 MWCO) concentrator. Elution solutions containing 1% acetic acid (pH 4.5), 8 M urea and salts were used in the size exclusion and ion exchange purification.

Surface Plasmon Resonance Analysis.

The receptor ECDs were purchased from R&D Systems (Minneapolis, USA) as C-terminal Fc-tagged chimera. The binding affinities were measured using a BIAcore 3000 (GE Healthcare, USA) following a protocol described previously by Allendorph et al. with minor modification (see, e.g., Allendorph et al., Plos One 6(11):e26402 (2011), and Allendorph et al., Proceedings of the National Academy of Sciences of the United States of America 103(20):7643-7648, 2006). In brief, the ECD-Fc proteins were prepared in a pH 4.0 solution and captured on flow cell 2, 3 and 4 of the CM5 sensor chip at a flow rate of 10 μL/min until the density researched ˜1,000 response units. Flow cell 1 was left blank for background subtraction. For kinetic analysis, purified MB109 was prepared in a series of 2-fold dilution in an assay solution (10 mM HEPES, pH 7.4, 150 mM NaCl, 3.4 mM EDTA and 0.005% Tween-20) and injected over the flow cells at a flow rate of 50 μL/ml. All tests were performed using a set of 2-fold diluted protein concentrations from 20 to 0.3 nM plus the assay solution as a blank. The data fitting was performed using a minimum of 4 concentrations and a global 1:1 Langmuir binding with mass transfer model.

Mammalian Cell Culture.

Hep3B, HepG2, AML12 and C2C12 cells were purchased from American Type Culture Collection. SNU-182, 354, 368 and 398 cells were purchased from KCLB (Korean cell line bank). Hep3B, HepG2 and C2C12 cells were cultured in Dulbecco's modified eagle's medium (DMEM) or RPMI1640 containing 100 U/mL penicillin, 0.1 μM streptomycin and 10% FBS. AML12 cells were cultured in DMEM/F-12 (1:1) containing 100 U/mL penicillin, 0.1 μM streptomycin, ITS™ premix (Becton Dickinson, USA) and 10% FBS. The cells were incubated at 37° C. under a humidified condition of 5% CO₂ and routinely subcultured using trypsine-EDTA when the cell density reached around 80% confluence. Cells were subcultured at 1:3˜5 every 3˜4 days so as to avoid changes in morphology/characteristics due to reaching confluence. All of the assays were done between passage 3˜12 after recovery from thawing.

General Luciferase Assays.

Cells were plated on 96-well plates (BD) in Opti-MEM low serum medium (Invitrogen) at 2×10⁴ cells/well and reverse co-transfected with Id1-De12-Luc and β-Galactosidase (β-Gal) plasmid using Fugene 6 (Promega). After 18 hours of transfection, cells were treated with ligands. After 24 hours of treatment exposure, cells were lysed using Luciferase lysis buffer (Promega) and luminescence was measured with plate luminometer (Berthhold, Bad Wildbad, Germany) by injecting D-Luciferin (Gold Biothechnology, MO, USA). Transfection variations were normalized by β-gal.

Smad1-Dependent Luciferase Reporter Assay.

Smad1-dependent luciferase assays were performed as previously described (see, Molecular Endocrinology 24(7):1469-1477 (2010), and Proceedings of the National Academy of Sciences 98(10):5868-5873 (2001)). In short, cultured C2C12 cells were trypsinized, washed once with PBS, resuspended in OptiMEM (Invitrogen, USA) plus 0.1% FBS and seeded in 96-well plates at 15,000 cells per 80 μL per well. Immediately after seeding, cells were transfected with the −1147Id1-luciferase plasmid, a Smad1 expression plasmid and a beta-galactosidase expression plasmid using Fugene6 (Promega, USA) according to the manufacturer's instruction. After 24 hours growth, 10 μL of series diluted protein ligands in OptiMEM medium were added in triplicate to the cell culture. After 16 hours of treatment, the cells were washed once with PBS and lysed for measuring luciferase and beta-galactosidase activities. The luciferase activity in each well was normalized for β-galactosidase activity, and the data were analyzed by using Prism 5 (GraphPad Software, Inc., USA). The commercial CHO-derived human BMP-9 was purchased from R&D Systems (Minneapolis, USA). The E. coli-derived BMP-2 was purchased from joint Protein Central (Incheon, Korea).

Cell Proliferation Assay.

HepG2, Hep3B and AML12 cells at around passage number 5-8 were trypsinized, washed once with PBS, re-suspended in low serum media, seeded in 96-well plates at 2,000 cells per 100 μL per well and incubated at 37° C. under a humidified condition of 5% CO₂. The low serum media included DMEM plus 1% FBS (for HepG2 and Hep3B cells) and DMEM/F-12 (1:1) plus ITS™ premix (Becton Dickinson, USA) and 1% FBS (for AML12 cells). Purified MB109 was prepared in the low serum media with a series of 10-fold dilutions. Twenty hours after seeding, 10 μL of the protein solutions were directly added into the cell culture in triplicate and incubated for an additional three days. The cell number was determined using the Cell Counting Kit-8 (CCK-8, Dojindo Laboratories, Japan) by directly adding 10 μL of CCK-8 reagent into the culture, incubating at 37° C. for 3 hours and measuring the absorbance at 450 nm using a microplate reader. Low serum media (without cells) was used as blank. The data was analyzed by using Prism 5.

Recombinant Proteins Ligands.

MB109 is a recombinant derivative of human BMP-9 produced from E. coli cells. MB109 contains a methionine residue in front of the mature form of human BMP-9 (Ser338-Arg429). Recombinant human BMP-2 was purchased from joint Protein Central (Incheon, Korea). Recombinant proteins were reconstituted just before use in 5 mM HCl (concentrations 0.8 mg/ml) and diluted in PBS for animal experiments or in cell culture medium for adipogenesis of hASC. The signaling activities of the recombinant proteins were determined by using cell-based assay luciferase activity. EC50s of BMP-2 and MB109 in C2C12 cells, transformed with Id1-Lux and Smad1 plasmids, were 28 ng/ml and 0.6 ng/ml, respectively. Human BMP-7 was purchased from R&D systems (Minneapolis, USA) and reconstituted in 5 mM HCl.

Adipogenesis of hASC.

Human ASCs were purchased from Invitrogen (Carlsbad, USA) and were grown in growth medium (MesenPro RS supplemented with 10 μg/ml Gentamicin and 2% FBS) to near confluency. After which, they were treated with various concentrations of ligands in the growth medium for 1 day, and induced for adipogenic differentiation for 7 days in the differentiation medium (growth medium plus 500 μM IBMX, 1 μM dexamethasone, 850 nM insulin, 125 nM indomethacin, 1 nM T3, and 1 μM rosiglitazone). For analysis of cAMP-enhanced expression of UCP1, cells were treated with vehicle or 100 μg/ml ligands in the growth medium for 1 day, differentiated for 7 days, stimulated with 0.5 mM dibutyryl cAMP (db-cAMP) for 6 hours, and subjected to RNA extraction. For confocal microscopic analysis of UCP1 expression, cells were grown on cover slide chambers treated with 100 μg/ml ligands in the growth medium for 1 day, differentiated in the differentiation medium for 10 days, and subjected to immunocytofluorescence staining.

Real-Time PCR.

Cells were plated on a 12-well plate (BD) at 2×10⁵ cells/well. After 24 hours, cells were treated with ligands. After 48 hours of treatment, RNA was extracted with TRIsure (Bioline, London, UK) according to the manufacturer's instruction. Synthesis of cDNA was performed using PrimeScript First Strand cDNA synthesis Kit (Takara) according to the manufacturer's instruction. Analysis of mRNA expression was determined with quantitative real-time polymerase chain reaction (qRT-PCR) using SYBR Premix Ex Taq II (Takara) with 0.4 μM of primers on PikoReal Real-Time PCR (Thermo). Abundance of mRNA in each sample was determined by the differences between the cycle threshold (CT) values for gene of interests and β-actin, ACT. Relative ratios of mRNA expression levels were defined as where ΔΔCτ=ΔCτₛₐₘₚₗₑ−ΔCτ_{control}, which reflect changes of mRNA expression levels from treated cells compared to those from untreated cells. All experiments were performed at least 3 times in triplicate. UCP-1 human and mouse primers were purchased from Qiagen (Venlo, Netherlands), product numbers PPH02223A (SEQ ID N0:37 and 38) and PPM05164B (SEQ ID N0:39 and 40), respectively. Sequences of primers for aP2, C/EBPα, PPARy, Cidea, CD137, Tmem 26, leptin, resistin, GLUT4, FAS, PEPCK and cyclophilin are shown in TABLE 1.

TABLE 1

| | Forward | Reverse | Species |
|---|---|---|---|
| β-actin | CTAAGGCCAACCGTGAAAAG | ACCAGAGGCATACAGGGACA | |
| | (SEQ ID NO: 3) | (SEQ ID NO: 4) | |
| p21 | CCAGCCTCTGGCATTAGAATTA | CGGGATGAGGAGGCTTTAAATA | |
| | (SEQ ID NO: 5) | (SEQ ID NO: 6) | |
| Survivin | GATGACGACCCCATAGAGGAAC | GGGTTAATTCTTCAAACTGCTTCT | |
| | (SEQ ID NO: 7) | (SEQ ID NO: 8) | |
| HBx | ACGGGGCGCACCTCTCTTTA | TGCCTACAGCCTCCTAGTAC | |
| | (SEQ ID NO: 9) | (SEQ ID NO: 10) | |
| AP2 | CATGTGCAGAAATGGGATGG | AACTTCAGTCCAGGTCAACG | H |
| | (SEQ ID NO: 11) | (SEQ ID NO: 12) | |
| Cyclophilin | CGAGGAAAACCGTGTACTATTA | TGCTGTCTTTGGGACCTTG | H |
| | G (SEQ ID NO: 13) | (SEQ ID NO: 14) | |
| C/EBP1α | TGGACAAGAGCAACGAG | TCATTGTCACTGGTCAGCTC | H |
| | (SEQ ID NO: 15) | (SEQ ID NO: 16) | |
| PPARγ | GAGCCCAAGTTTGAGTTTGC | GCAGGTTGTCTTGAATGTCTTC | H |
| | (SEQ ID NO: 17) | (SEQ ID NO: 18) | |
| CD137 | CCTGTGATAACTGTCAGCCTG | TCTTGAACCTGAAATAGCCTGC | M |
| | (SEQ ID NO: 19) | (SEQ ID NO: 20) | |
| CIDEA | ATCACAACTGGCCTGGTTACG | TACTACCCGGTGTCCATTTCT | M |
| | (SEQ ID NO: 21) | (SEQ ID NO: 22) | |
| Cyclophilin | CAGACGCCACTGTCGCTTT | TGTCTTTGGAACTTTGTCTGCAA | M |
| | (SEQ ID NO: 23) | (SEQ ID NO: 24) | |
| FAS | CCCCTCTGTTAATTGGCTCC | TTGTGGAAGTGCAGGTTAGG | M |
| | (SEQ ID NO: 25) | (SEQ ID NO: 26) | |
| GLUT4 | GATTCTGCTGCCCTTCTGTC | ATTGGACGCTCTCTCTCCAA | M |
| | (SEQ ID NO: 27) | (SEQ ID NO: 28) | |
| Leptin | GTGCCTATCCAGAAAGTCCAG | TGAAGCCCAGGAATGAAGTC | M |
| | (SEQ ID NO: 29) | (SEQ ID NO: 30) | |
| PEPCK | CCATCCCAACTCGAGATTCTG | CTGAGGGCTTCATAGACAAGG | M |
| | (SEQ ID NO: 31) | (SEQ ID NO: 32) | |
| Resistin | TCCTTTTCTTCCTTGTCCCTG | GACTGTCCAGCAATTTAAGCC | M |
| | (SEQ ID NO: 33) | (SEQ ID NO: 34) | |
| Tmem26 | GCACCATCACTAGAGACCAAC | ACAAGAATGCCAGAGACCAG | M |
| | (SEQ ID NO: 35) | (SEQ ID NO: 36) | |

ELISA.

Serum levels of Leptin were determined using the Quantikine ELISA (R&D Systems) as described in the manufacturer's instructions. Concentrations were calculated using a standard curve generated by Leptin standards included in the kit.

MTT Assay.

Cells were plated on a 96-well plate (BD (NJ, USA)) at 4×10³ cells/well. After 18˜24 hours, cells were treated with indicated concentration of different ligands. After desired period of exposure, 3-(4,5-dimethylthiazol-2-yl)-2,5-diphenyltetrazolium bromide (MTT) reagent (5 mg/ml in PBS, Sigma) was added and incubated at 37° C. until purple precipitation was visible. MTT crystal was dissolved in 4 mM HCl, 0.1% NP-40 in isopropanol for 15 minutes and absorbance was measured at 590 nm and baseline corrected at 700 nm.

Western Blot.

Cells were plated on a 6-well plate (BD) at 2×10⁵ cells/well. Cells were treated with ligands for 2 days and lysed using cell lysis buffer (Cell Signaling, MA, USA) with 1 mM PMS and phosphatase inhibitor cocktail (Roche). Cell lysate's total protein amount was quantified using Bradford assay. Proteins were separated on SDS-polyacrylamide gels and transferred onto nitrocellulose membrane (GE healthcare (NJ, USA)). Further processing of the Western blot was performed according to the manufacturer's instruction.

Cell Cycle Arrest Analysis.

Cells were plated on a 6-well plate (BD) at 2×10⁵ cells/well. After 12 to 18 hours, cells were serum starved for 24 hours and changed back to 10% heat inactivated charcoal stripped FBS media along with ligand treatment. Cells were exposed to ligands for 2 days and harvested for fixation in 80% ethanol 20% DPBS/0.1% BSA. Cells were fixed in 4° C. for 24 hours and stained with a propidium iodide solution (sigma) containing RNAse A. Stained cells were analyzed with BD LSRII FACScan system using FACS Diva software (BD). Acquired data was analyzed by using Flowing Software (Perttu Terho, University of Turku, Finland).

Animals.

C57BL/6 (male, 8 week old) mice were randomly assigned to normal chow (NC) diet groups (NC/sham, NC/BMP-2, NC/MB109) and 60% Kcal high fat (HF) diet groups (HF/sham, HF/BMP-2, HF/MB109). Each cage housed 4 mice. Each mouse received vehicle (PBS) or 100 μg/kg recombinant human BMP-2 or MB109 twice a week for 8 weeks. Food consumption and body weights of mice were recorded every week. Blood glucose levels after 16 hour fasting were measured every 4 week.

RNA Extraction from Fat Tissues.

Fat tissues were snap frozen in liquid nitrogen and used to extract RNA using Trisure (Bioline, UK) according to the manufacturer's instruction with only minor modifications. Briefly, fat tissues were minced in Trisure (˜0.03 cm² fat tissue/ml Trisure) using a motorized hand pestle and centrifuged at 13000×g for 5 min. The lipid layer was removed and the layer containing Trisure solution was used to isolate RNA.

Histological Analysis.

Fat tissues were processed for paraffin embedding and 6 micron tissue sections on slide glasses were processed for hematoxylin-eosin staining or DAB staining using ImmPRESS detection system (VECTOR Lab, USA) according to the manufacturer's instruction, and analyzed under a microscope. Anti-UCP1 antibody was purchased from Abcam (Cambridge, England).

Expression and Purification of Denatured, Monomeric MB109.

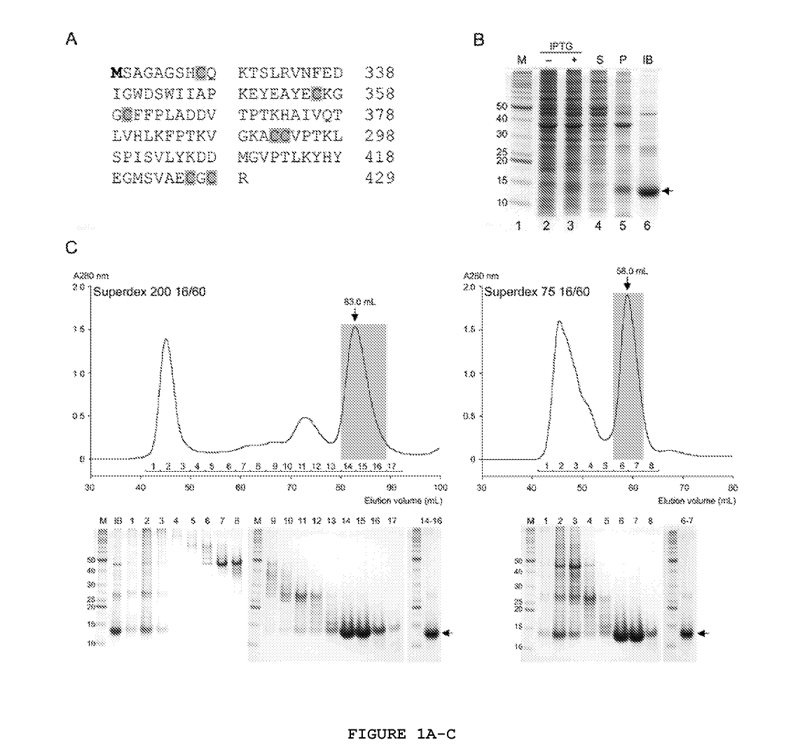

To express the mature domain of human BMP-9, a synthetic, codon-optimized gene, encoding Ser320-Arg429 of NCBI Gene ID:2658 (see FIG. 1A) was cloned into a pET21a vector downstream from a T7 promoter. This gene product, is referred to as MB109 polypeptide, contains Met at the N-terminus followed by the coding region of the mature region of human BMP9 (Ser320-Arg429). The expression plasmid of MB109 was transformed into BL21 E. coli cells and cultured in LB-broth under aerobic conditions in a shaker incubator. After inducing with IPTG for 20 hours at 37° C., the cells were lysed by microfluidization and the expression of the target polypeptide was analyzed by SDS-PAGE. In the whole cell lysate, MB109 polypeptide is present in the non-soluble fraction and can be separated by centrifugation, indicating the formation of an inclusion body (see FIG. 1B, lane 5). After washing for three times, some cellular proteins remain associated with the isolated inclusion body (see FIG. 1B, lane 6). About 150 mg of the isolated inclusion body was obtained from one liter of cell culture.

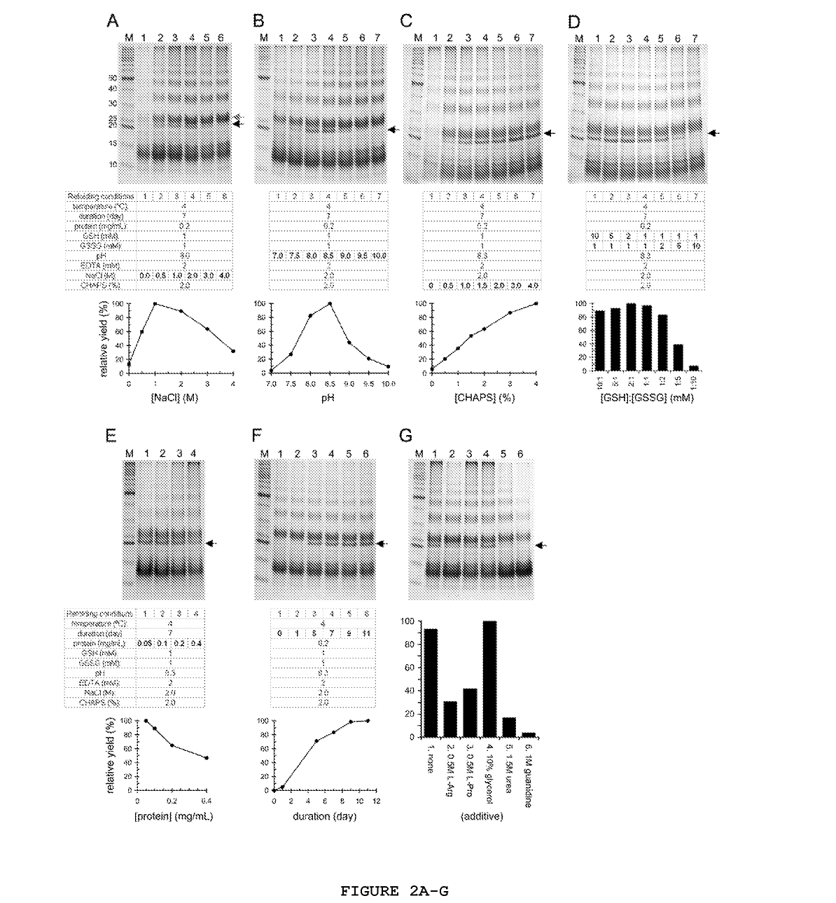

To further improve the purity of the MB109 polypeptide for refolding, the isolated inclusion body was solubilized in reduced and denaturing conditions and subjected to chromatographic purification by size exclusion chromatography using a HiLoad Superdex™ 75 or 200 columns (GE Healthcare). It was found that even though these two types of gel filtration columns have different separation ranges of molecular size, they gave similar results in terms of final purity and yield of the denatured monomeric MB109 polypeptide. In Superdex™ 200 column, the solubilized inclusion body was eluted as three major peaks at around 46, 72 and 83 mL, with the target protein present in the last peak (see FIG. 1C, gray bar). In Superdex™ 75 column, the proteins came out in two well-separated peaks, with the target protein present in the second peak (see FIG. 2D, gray bar). Therefore, the fractions containing the monomeric MB109 polypeptide were pooled and concentrated for setting up refolding. About 50 mg of monomeric MB109 polypeptide was purified from 100 mg of solubilized inclusion body by using either column.

Effect of Salt Concentration on the Refolding of MB109.

The buffer solutions, which readily refold DPP and BMP-2, 3, 6, 2/6, 12 and 13, contain a minimal set of six components: detergent (CHAPS), salt (NaCl), chelator (EDTA), redox agents (reduced and oxidized glutathiones) and buffer (Tris-HCl). To see which components are useful for refolding MB109 polypeptide and to identify their optimal values for maximal refolding yield, the NaCl concentration was varied from 0 to 4M and the other refolding variables fixed at their commonly used values, including 2.0% CHAPS, 2 mM EDTA, 1/1 mM GSH/GSSG, 50 mM Tris-HCl at pH 8.0, 0.2 mg/mL protein concentration and incubating the samples at 4° C. for 7 days (see FIG. 2A middle panel). The refolding results were directly visualized by non-reduced SDS-PAGE. In the absence of NaCl, most protein aggregates in the refolding solution, so there were not much protein remaining in the supernatant after centrifugation (see FIG. 2A top panel, lane 1). In the presence of NaCl, MB109 polypeptides folds into stable monomers, dimers and multimers (see FIG. 2A top panel, lanes 2-6).

In FIG. 2A top panel, there are two visually well-separated bands in refolding conditions 2-6, which have a size close the theoretical molecular weight of dimeric MB109 protein (24.4 kDa). The protein of the bottom dimer band (black arrow) was purified, and its bioactivity confirmed as a “functional dimer” (i.e., correctly refolded MB109 protein). The upper dimer bands (gray arrow), which contains no bioactivity after being purified, is referred as ‘chemical dimer’ hereafter for discussion purpose. The densitometry of the functional dimer bands in the SDS-PAGE image (see FIG. 2A, bottom panel) reveals that the optimal refolding salt concentration for MB109 protein is around 1.0-2.0 M. When the salt concentration is above 2M, the refolding efficiency is gradually reduced.

Effect of pH on the Refolding of MB109.

To analyze the pH effect on refolding MB109, the salt concentration was then fixed at 2M and the buffer pH was varied between 7.0 and 10.0 with a 0.5 interval. As shown in FIG. 2B upper panel, the functional dimer band was only observed at pHs from 7.5 to 9.5 (lanes 2-6), whereas the chemical dimmers, as well as other multimers and monomers, were present in all tested pHs. The refolding yield of the functional dimer has sharp pH dependency. The optimal refolding pH is between 8.0 and 8.5, and the refolding efficiency reduces drastically when the pH is beyond this range (see FIG. 2B, bottom panel).

Effect of Detergent Concentration on the Refolding of MB109.

In the standard refolding conditions, 1.8-2% CHAPS has been used to refold DPP, BMP-2, 3, 6, 2/6, 12 and 13. To test the effect of CHAPS on refolding MB109, 0 to 4% CHAPS was used in the refolding solution. As shown in FIG. 2C top panel, MB109 protein forms visible aggregates in the refolding solution after 7 days of incubation in the absence of the detergent, so there were not much protein remaining in the supernatant (lane 1). At a CHAPS concentration above 0.5%, the functional dimer can be refolded and the refolding efficiency is positively correlated with the amount of CHAPS in the refolding solution (see FIG. 2C bottom panel). When compared to the commonly used 2% CHAPS, the refolding yield increases about 37% and 58% in the presence of 3% and 4% CHAPS, respectively,

Effect of Redox Conditions on the Refolding of MB109.

Since a bioactive BMP molecule contains several disulfide bonds, the reduced and oxidized glutathiones (GSH and GSSG, respectively) have been used as a redox system in refolding solution to allow the formation and reshuffling of disulfide bonds. To identify the optimal redox condition, a millimolar ratio of between 10:1 and 1:10 of GSH to GSSG was tested (see FIG. 2D). Interestingly, the refolding efficiency of the functional dimer depends mostly on the amount of GSSG, but not GSH, in the refolding solution. In other words, the more oxidizing power (GSSG) in the solution, the less efficient the functional dimer can be refolded (see FIG. 2D, conditions 4-7). In contrast, increasing the reducing power (GSH) in the refolding solution does not affect the refolding yield much (see FIG. 2D, conditions 1-4). The maximal refolding yield was observed at the combination of 2 mM GSH and 1 mM GSSG.

Effect of Protein Concentration on the Refolding of MB109.

In all of the refolding conditions tested, MB109 is prone to form a chemical dimer and higher order of multimers. The results suggest that the protein concentration (0.2 mg/mL) used for the refolding tests may be too high to cause the non-specific multimerization and affect the refolding efficiency of the functional dimer. To see how protein concentration affects the refolding yield, between 0.05 and 0.4 mg/mL of protein concentration was tested (see FIG. 2E). Indeed, the refolding yield is inversely correlated with the protein concentration in the refolding solution. At 0.05 mg/mL, the tendency of forming higher order multimers is significantly reduced (see FIG. 2E top panel, lane 1), and the yield of the functional dimer is increased about 55% as compared to the refolding at 0.2 mg/mL (bottom panel).

Refolding Duration Vs Refolding Yield.

In addition to the chemical variables tested above, the refolding time course was also analyzed for up to 11 days at 4° C. to identify the time required to achieve maximal refolding yields. As shown in FIG. 2F, the functional dimer starts to be refolded after 2 days and the yield reaches plateau after 9 days of incubation.

Effect of Secondary Additive on the Refolding of MB109.

Some chemical additives are known to be effective folding aids for in vitro protein refolding. To see whether the refolding yield could be further improved, the commonly used aggregation suppressors and denaturants, including L-arginine, L-proline, glycerol, urea and guanidine were examined. As shown in FIG. 2G, the addition of 0.5 M L-arginine, 0.5 M L-proline, 1.5 M urea and 1 M guanidine significantly reduced the efficiency of refolding MB109, whereas the addition of 10% glycerol had little or slightly better effect on the refolding yield.

Effect of Host Cell Contaminants on the Refolding of MB109.

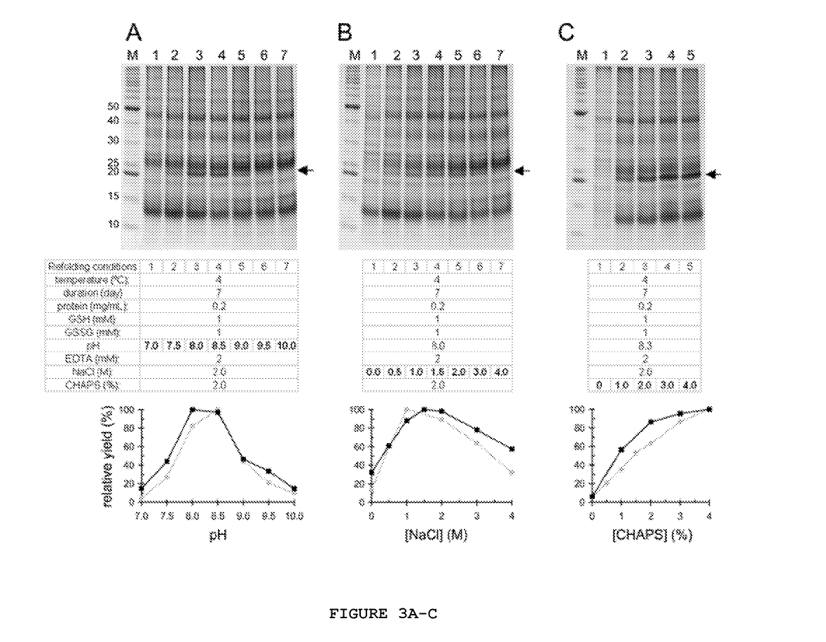

As shown in FIG. 1B, lane 6, the isolated inclusion body of MB109 contains visible amount of host cell contaminants on the SDS-PAGE, although these contaminants can be effectively removed by size exclusion chromatography, it would be more cost-effective if this purification step can be omitted. Therefore, the refolding efficiency was tested in the presence of the cell contaminants by directly using the isolated inclusion body to set up the refolding tests in different pHs and concentrations of salt and detergent. As shown in FIG. 3 upper panels, the functional dimer (black arrows) can be refolded in the presence of host cell contaminants. Similar to the size-exclusion purified inclusion body, MB109 in the presence of the host cell contaminants has an optimal refolding pH of 8.0-8.5 (see FIG. 3A), and an optimal refolding NaCl concentration of 1-2M (see FIG. 3B). In the CHAPS detergent, its refolding yield also positively correlated with the detergent concentration, and reaches plateau at around 3% (see FIG. 3C).

Purification of the Refolded MB109 Protein.

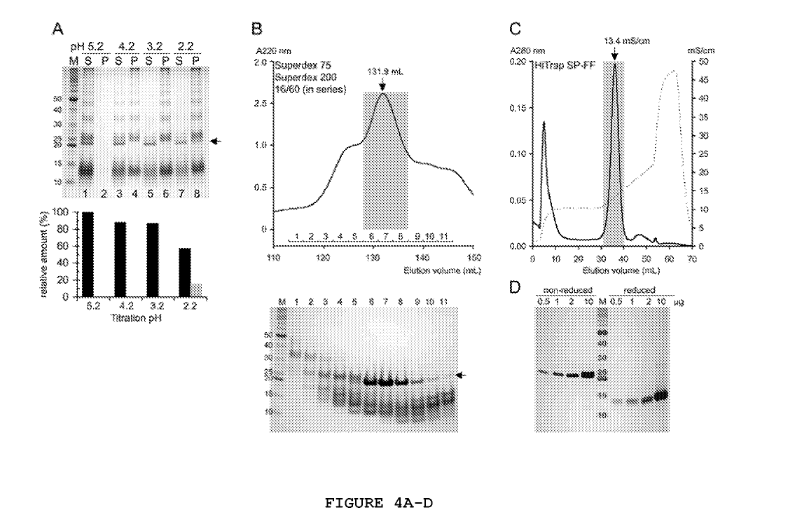

In all of the refolding conditions that were analyzed, the denatured MB109 can be refolded not only into the functional dimer, but also other disulfide variants, including the chemical dimer, monomers and multimers (see FIG. 2). These disulfide variants are considered as contaminants in the purification stage, and need to be removed. To separate the functional dimer from the contaminants, it was determined that the majority of the contaminants can be effectively separated from the functional dimer by precipitation when the pH of the refolding solution was directly titrated to 3.2 with acetic acid. As shown in FIG. 4A, the contaminants form water insoluble aggregate at pH 4.2 and 3.2 and can be precipitated by centrifugation (lanes 4 and 6, respectively), while the functional dimer remains in the supernatant (lane 3 and 5). At pH 2.2, however, the functional dimer starts to form water insoluble aggregates as well (see FIG. 4A lane 8, black arrow).

After acidic fractionation, some monomeric contaminants and small amounts of chemical dimer and multimers remain in the supernatant (see FIG. 4A, lane 5). To further purify the functional dimer, size exclusion chromatography with Superdex 75 and Superdex 200 connected in series were used. As shown in FIG. 4B, the multimers and chemical dimers are eluted at 113-128 mL before the function dimer was eluted at 128-137 mL (gray box). However, some of the monomeric disulfide variants were co-eluted with the functional dimer (see FIG. 4B bottom panel, lane 6-8).

To remove the remaining monomeric disulfide variants, SP Sepharose Fast Flow cation exchanger was found to be effective. As shown in FIG. 4C, the majority of the monomeric contaminants can be washed out at ˜10.5 mS/cm and the functional dimer can then be eluted around 13.4 mS/cm. The purity of the purified functional dimer was greater than 95%, as determined by non-reduced and reduced SDS-PAGE (see FIG. 4D). The final yield of the purified MB109 protein was from 7.8 mg to 100 mg when refolded under the optimal conditions.

Bioactivity of Refolded MB109 Protein.

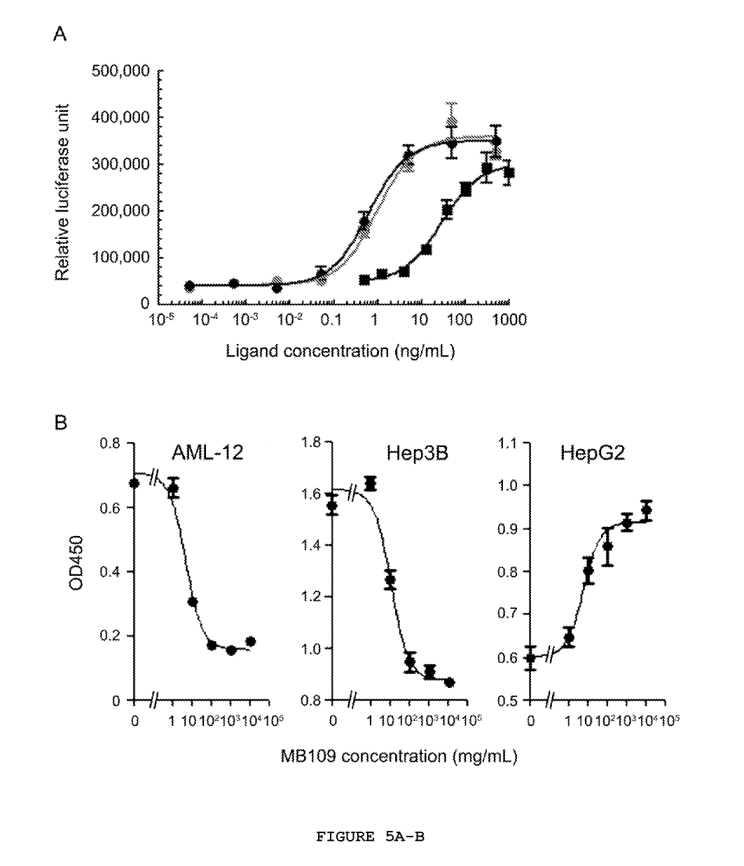

The bioactivity of the purified MB109 protein was first tested by examining its ability to stimulate the Smad1 signaling pathway using an established Smad1-dependent luciferase reporter system in the mouse myoblast C2C12 cells. A CHO-derived BMP-9 and an E. coli-derived BMP-2 were used as positive controls. As shown in FIG. 5A, the purified MB109 protein is able to induce dose-dependent Smad1 signaling response with an EC50 of 0.61 ng/mL (25 μM, black circles), which is similar to that induced by CHO-derived BMP-9 (EC50 0.92 ng/mL, gray circles), and is about 43-fold stronger than that induced by E. Coli-derived BMP-2 (EC50 28.1 ng/mL or 1.08 μM, black squares).

Since BMP-9 has been shown to be predominantly expressed in liver cells, the purified MB109 protein on different liver cell lines was also tested to see if it would affect cell proliferation. Cells were treated with a series of 10-fold dilutions of MB109 and then the cell numbers were determined three days post treatment. Interestingly, MB109 inhibited the proliferation of mouse normal hepatocyte AML-12 cells (see FIG. 5B left panel) and human hepatoma Hep3B cells (middle panel) in a dose-dependent manner. The IC₅₀ are 4.9 and 12.4 ng/mL, respectively. In contrast to AML-12 and Hep3B, MB109 promoted the proliferation of another human hepatoma cell line, HepG2, in a dose-dependent manner with an EC50 of 6.1 ng/mL (see FIG. 5B, right panel).

To determine receptor binding specificity of MB109, purified MB109 was subjected to surface plasmon resonance analysis to determine its binding affinities to immobilized extracellular domains (ECDs) of Type I and Type II receptors, including ALK1, ActRIa (ALK2), ActRIb (ALK4), BMPRIa (ALK3), BMPRIb (ALK6), TGFβRI (ALK5), ALK7, ActRIIa, ActRIIb, BMPRII, TGF-βRII and MISRII. Among these receptor ECDs, MB109 had strong binding affinity only to ALK1, ActRIIb and BMPRII, while the binding to the other receptor ECDs were either very transient (to ActRIIa) or non-detectable (see TABLE 2).

TABLE 2
Binding affinities of MB109 as determined
by surface plasma resonance.

| ECD-Fc | k_{off} (s⁻¹)/kₒₙ (M⁻¹s⁻¹) | K_{D} (pM) |
|---|---|---|
| TYPE 1 RECEPTOR: | | |
| ALK1 | 8.84 × 10⁻⁴/4.24 × 10⁶ | 209 |
| ActRIa (ALK2) | ND | ND |
| BMPRIa (ALK3) | ND | ND |
| ActRIb (ALK4) | ND | ND |
| TGF-betaRI (ALK5) | ND | ND |
| BMPRIb (ALK6) | ND | ND |
| ALK7 | ND | ND |
| TYPE II RECEPTOR: | | |
| ActRIIa | 1.21 × 10⁻²/2.85 × 10⁶ | 4,270 |
| ActRIIb | 1.52 × 10⁻³/9.91 × 10⁶ | 154 |
| BMPRII | 1.53 × 10⁻³/4.63 × 10⁶ | 330 |
| TGF-βRII | ND | ND |
| MISRII | ND | ND |

ECD-Fc, extracellular domain with C-terminal human IgG1 tag. ND, non-detectable (no signal above the background detection was detected in all tested protein concentration ranging from 20 to 0.3 nM).

MB109 Protein Inhibits the Growth of Certain Hepatocellular Carcinoma Cells.

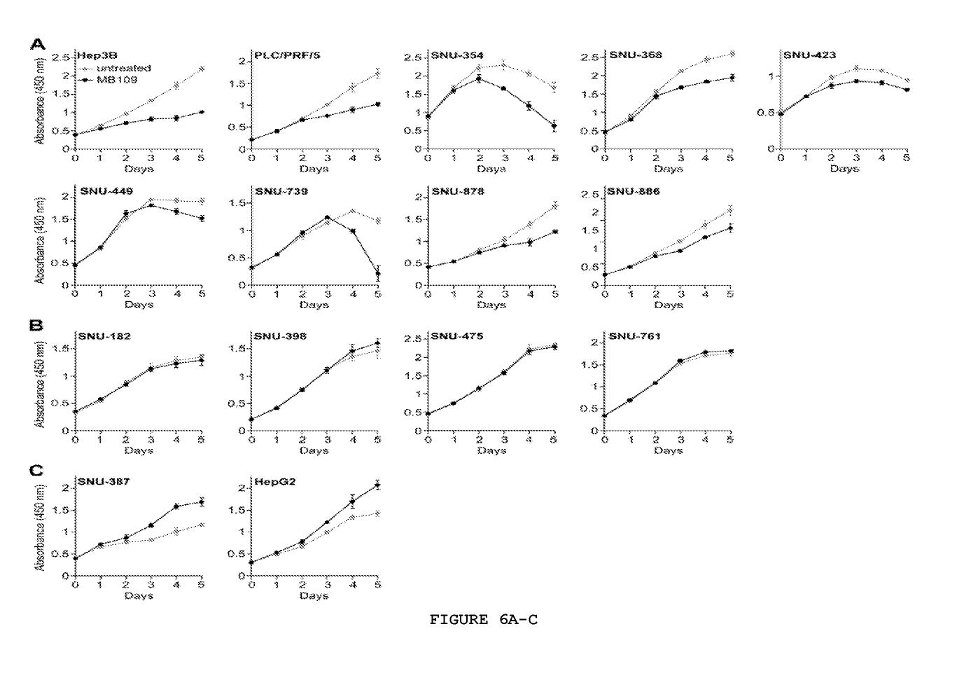

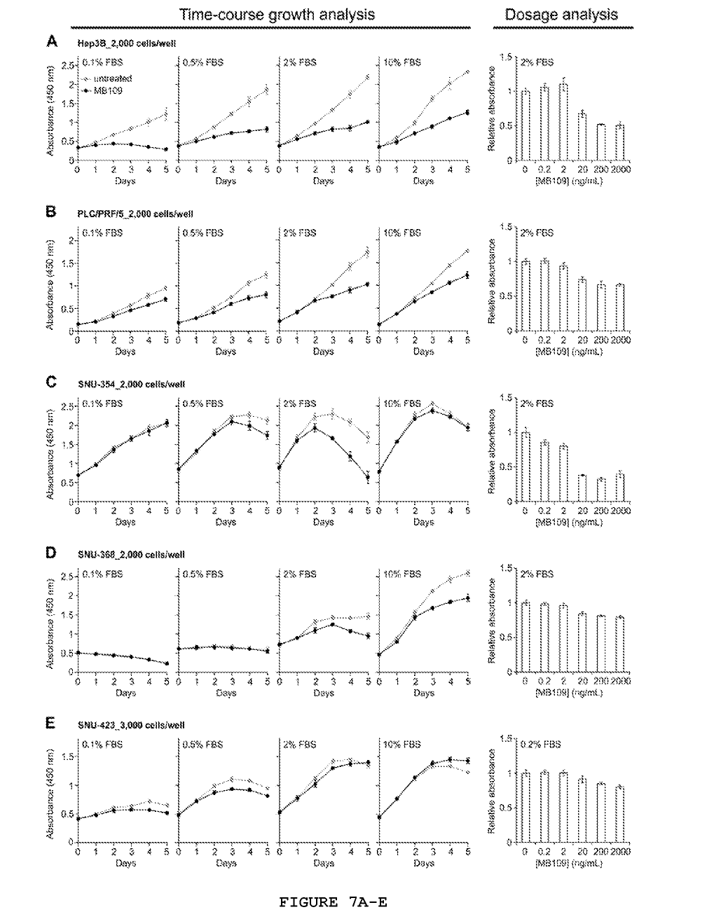

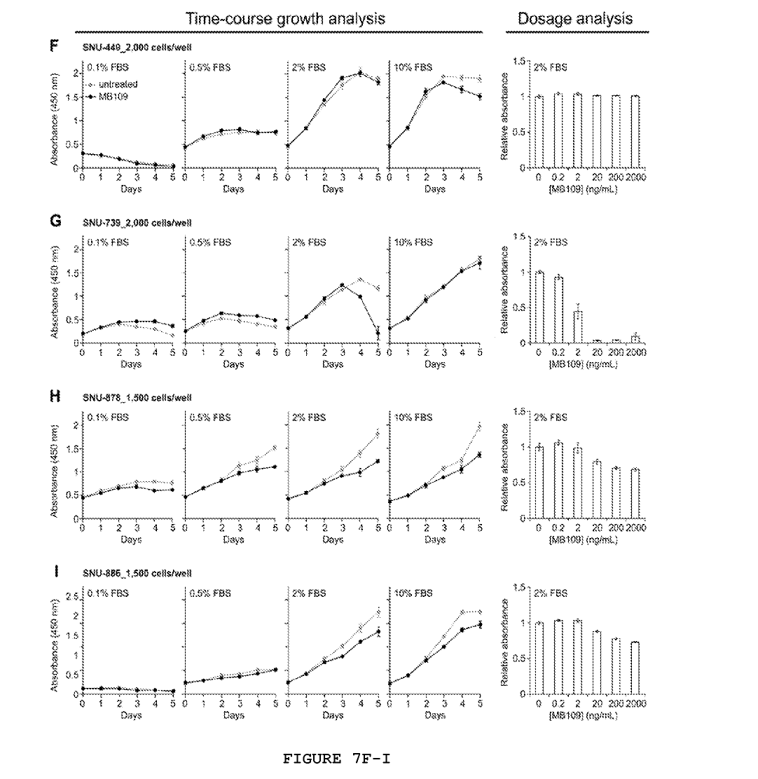

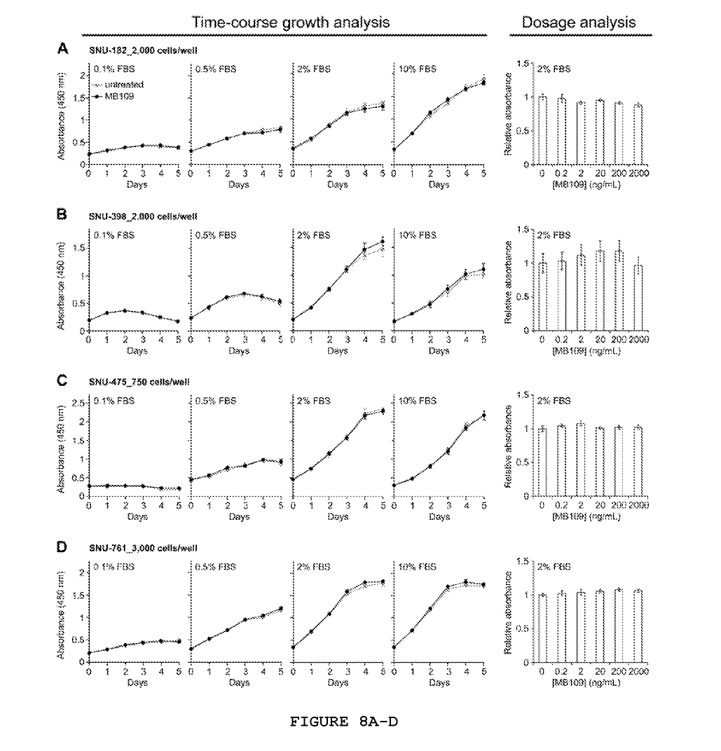

To study the effects of BMP-9 signaling on HCC cell growth, fifteen HCC cell lines were tested for proliferation in vitro using MB109. The cells were treated with 200 ng/mL of MB109 and the cell numbers were determined by a MTT assay during 5-day treatment (FIG. 6). The effect of serum concentration on cell growth was also analyzed in parallel by culturing the cells at 0.1, 0.5, 2 and 10% FBS separately (FIGS. 7-9). Dosage analysis was carried out using MB109 concentrations between 0.2 and 2,000 ng/mL (FIGS. 7-9). As shown in FIG. 6A, 200 ng/mL of MB109 treatment significantly inhibited the growth of nine HCC cells including Hep3B, PLC/PRF/5, SNU-354, SNU-368, SNU-423, SNU-449, SNU-739, SNU-878 and SNU-886. Four other cells, SNU-182, SNU-398, SNU-475 and SNU-761, did not respond to MB109 treatment (FIG. 6B), and the other two cells, SNU-387 and HepG2, were promoted by MB109 treatment (FIG. 6C). Dosage analysis revealed that the effective MB109 concentrations to cause the inhibitory or promotional responses were around 2-20 ng/mL and above (FIGS. 7 and 9). These results reveal that the growth of a certain subset of HCC cells can be effectively inhibited by exogenous MB109 treatment.

MB109 Protein Induces p21 Expression and Survivin Suppression Causes Cell Cycle Arrest.

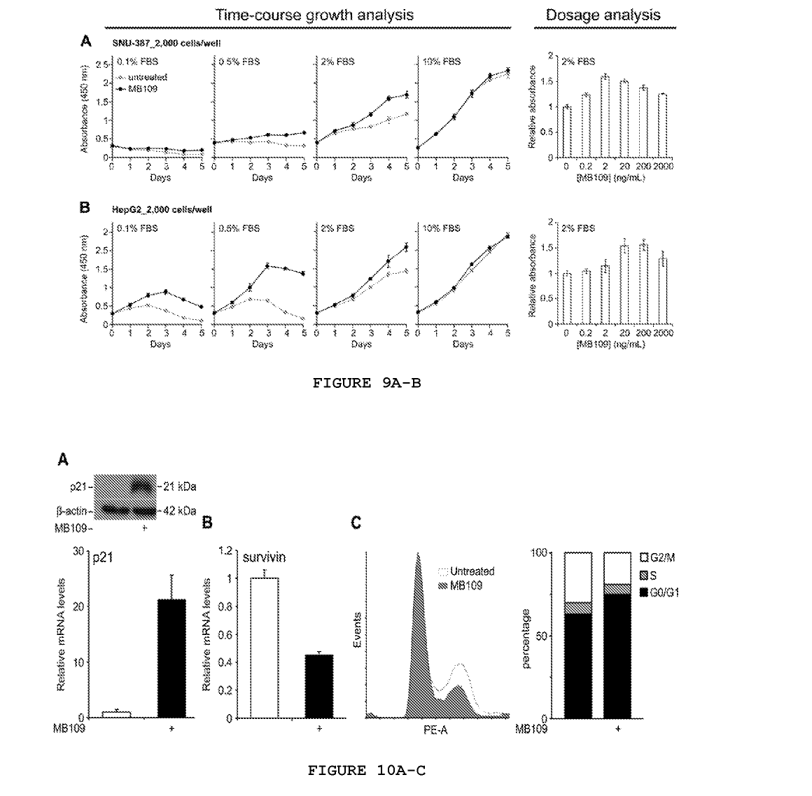

To identify molecular mechanism of the MB109-induced anti-proliferative effect, Hep3B cells p21 mRNA and protein expression levels were analyzed using real-time (RT) PCR and western blot analyses. When Hep3B cells were exposed to 200 ng/mL of MB109 for 24 hours, p21 expression was dramatically induced at both the mRNA and protein levels (FIG. 10A). MB109 also down regulated the level of survivin mRNA in Hep3B cells as examined by RT-PCR (FIG. 10B). Survivin is an oncogene and the result is consistent with a previous report on HepG2 cells, where survivin expression was also regulated by p21 overexpression (Xiong et al., 2008). Because p21 and survivin are key components of cell cycle regulation, the cell cycle status of Hep3B cells was examined over 48 hours of MB109 treatment at 200 ng/mL. The MB109 treatment significantly increased G0/G1 and decreased S and G2/M populations of the cells (FIG. 10C). These results provide a possible explanation for a direct correlation among MB109-induced growth inhibition, p21 induction, survivin suppression and G0/G1 cell cycle arrest, implicating that the downstream effectors of the BMP-9 signaling pathway in Hep3B cells include p21 and survivin to exert G0/G1 cell cycle arrest.

ID3 is Involved in MB109-Induced p21 Expression in Hep3B Cells.

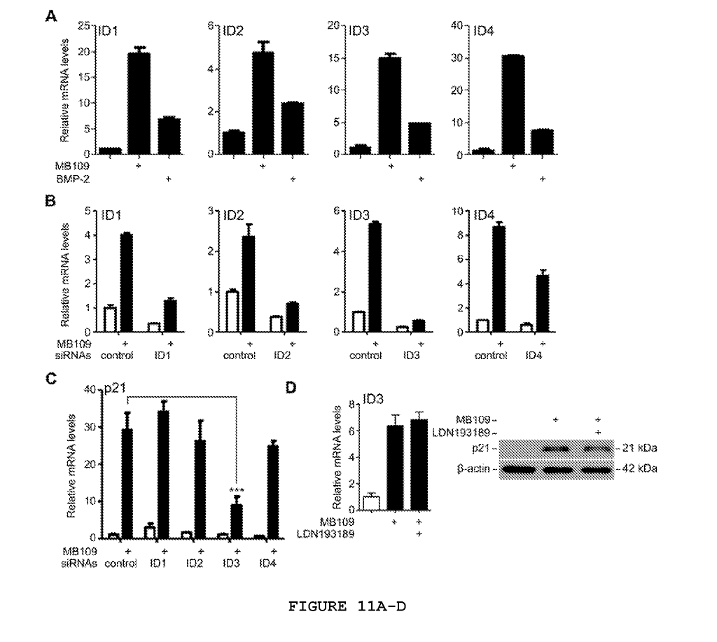

To further investigate how MB109 may induce p21 expression, analysis of an inhibitor of DNA binding (ID) proteins, ID1, ID2, ID3 and ID4 was examined. IDs are dominant negative basic Helix-Loop-Helix (bHLH) family proteins, which bind on bHLH transcription factors to inhibit their binding to DNA and thus play a role in cell cycle regulation and oncogenesis (Norton, 2000; Perk et al., 2005). MB109 treatment induced strong mRNA expression of all four IDs in Hep3B cells (FIG. 11A). To identify which IDs are involved in MB109-induced p21 expression, knocked-down (KD) was performed of each ID individually using siRNA, so that the basal and MB109-induced mRNA expression were independently reduced (FIG. 11B). While the expression of all IDs was effectively knocked down, only the ID3-KD cells had significantly attenuated p21 expression upon MB109 treatment (FIG. 11C).

The involvement of ALK2, 3 and 6 type I receptors in the MB109-induced p21 expression were tested by a chemical inhibitor, LDN193189, to block the signaling capability of ALK2/3/6 in order to study their involvement in MB109-induced ID3 and p21 expression. As shown in FIG. 11D, blocking ALK2/3/6 had little effect on ID3 mRNA expression (left panel) and p21 protein expression (right panel) upon MB109 treatment. These results demonstrate that ID3 mediates the induction of p21 expression in the BMP-9 signaling pathway, which does not involve ALK2/3/6 type I receptors.

p38 MAPK Controls MB109-Induced ID3 and p21 Expression in Hep3B Cells.

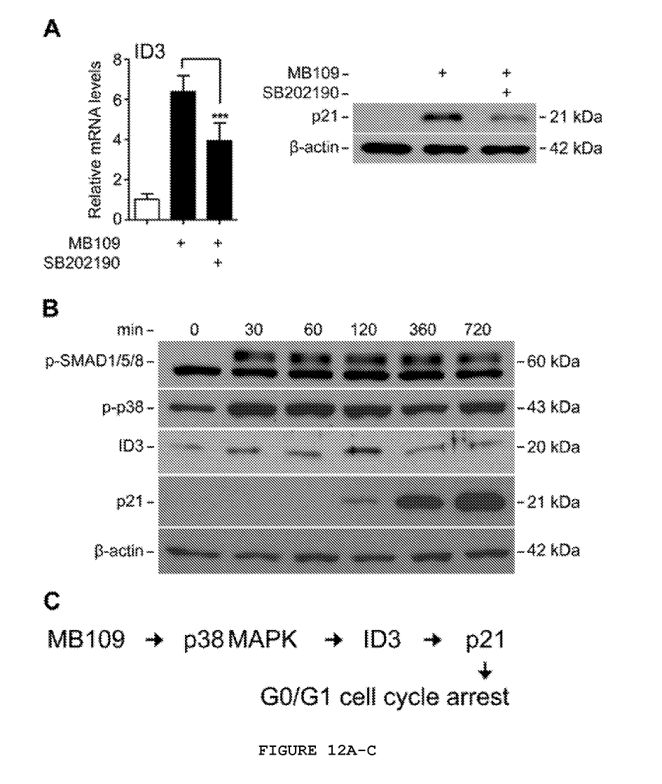

To identify the upstream signaling molecules that induce ID3 expression, analysis of p38 mitogen-activated protein kinase (p38 MAPK), which is known to transduce SMAD-independent TGF-β signaling in various cell types (Miyazono et al., 2005), was performed. A chemical inhibitor, SB202190, was used to block p38 MAPK activity in Hep3B cells. The expression of ID3 mRNA and p21 protein was analyzed by RT-PCR and western blot, respectively. As shown in FIG. 12A, blocking the activity of p38 MAPK suppressed MB109-induced ID3 mRNA (left panel) and p21 protein expression (right panel). These results demonstrate that the MB109-induced ID3/p21 expression in Hep3B cells requires p38 MAPK activity.

Analysis of the time courses of the expression of the major signaling components by western blot over 720 minutes (12 hours) of MB109 treatment was also performed. As shown in FIG. 12B, p38, as well as SMAD1/5/8, was phosphorylated efficiently within 30 minutes after MB109 treatment. ID3 expression peaked at about 120 minutes, and then followed by dramatic induction of p21 expression. These data demonstrate that the MB109-induced G0/G1 cell cycle arrest in Hep3B is through p38/ID3/p21 signaling pathway, independent from SMAD signaling (FIG. 12C).

Prolonged MB109 Treatment Reduces Cancer Stem Cell Population in Hep3B Cells.

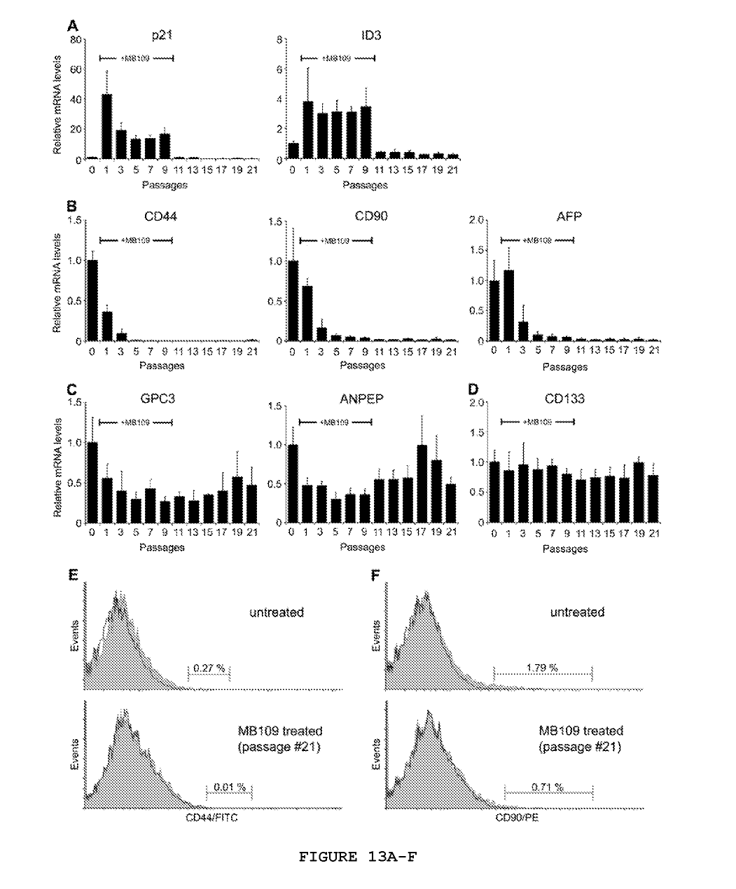

Recently, it has been reported that ID1 and ID3 govern self-renewal and cell cycle restriction on cancer stem cell (CSC) populations of colon cancer through regulation of p21 (O'Brien et al., 2012). Because of the specific signaling pathway of BMP-9 through ID3 leading to p21 expression to suppress the growth of Hep3B cells, the effect of MB109 on the CSC population of these cells was examined. To investigate whether BMP-9 signaling plays a direct role on differentiating liver cancer stem cells (LCSCs), Hep3B cells were grown and subcultured in medium containing 200 ng/ml of MB109 for 10 passages. The ligand was then removed from the growth medium at passage 11 and the cells were continued to be grown and subcultured for another 11 passages. The mRNA level of p21, ID3 and six prominent LCSC markers, including CD44, CD90, alpha-fetoprotein (AFP), glypican 3 (GPC3), alanine aminopeptidase (ANPEP or CD13) and CD133 (Haraguchi et al., 2010; Ho et al., 2012; Liu et al., 2011), were monitored every other passage by RT-PCR. FIG. 13A, left panel, shows that p21 expression levels were peaked about 40-fold after MB109 treatment (passage #1) and stabilized at around 15-fold following the prolonged MB109 treatment (passages #3-9). Upon removal of MB109, p21 expression levels dropped back immediately to the original levels (passage #11). ID3 expression levels also increased during the course of MB109 treatment, and returned to basal level right after the ligand was removed (FIG. 13A, right panel). In FIG. 13B, more than 10-fold reduction was observed on the expressions of CD44 (left panel), CD90 (middle panel) and AFP (right panel) during the prolonged MB109 treatment. Upon removal of MB109, the expression of these three LCSC markers remained at reduced levels toward the end of the experiment. In FIG. 13C, expression levels of GPC3 (left panel) and ANPEP (right panel) were moderately reduced by prolonged MB109 treatment and gradually increased after MB109 was removed. In FIG. 13D, the prolonged MB109 treatment had little or slightly suppression effect on CD133 expression. To determine whether the reduced CD44 and CD90 mRNA levels resulted in the actual reduction of CD44′ and CD90′ cell populations, the MB109-treated cells at passage #21 were analyzed by flow cytometry. Indeed, MB109 treatment caused reduction of CD44′ population from 0.27% to 0.01% (FIG. 13E) and reduction of CD90′ population from 1.79% to 0.71% (FIG. 13F). All together, these results demonstrate that MB109 is able to permanently turn off the expression of CD44, CD90 and AFP, which could be due to differentiation of CD44′, CD90′ and AFP′ populations into another CD44⁻, CD90⁻ and AFP⁻ cell types.

MB109 Suppresses Hep3B Cell Growth and LCSC Population in Mouse Xenograft Model.

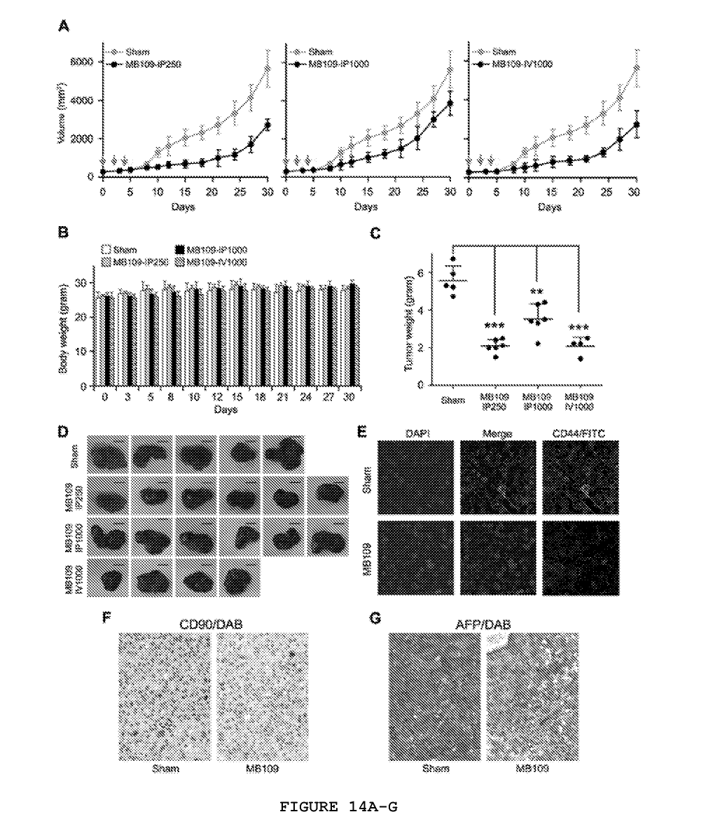

Because MB109 was able to induce an anti-proliferative effect and LCSC suppression on HCC cells in vitro, xenograft experiments were carried out to examine these effects in vivo. Hep3B cells were xenografted into non-obese diabetic/severe combined immunodeficiency (NOD/SCID) mice to form tumors of about 300 mm³, and then the mice were randomly assigned to 4 groups: Sham, MB109-IP250, MB109-IP1000 and MB109-IV1000. Three single-dose injections were made either intraperitoneally (250 or 1000 μg/kg body weight for MB109-IP250 and MB109-IP1000 groups, respectively) or intravenously (1000 μg/kg for the MB109-IV1000 group) at day 0, 2 and 4. In FIG. 14A, MB109 injections inhibited the tumor growth in all three experimental groups. The inhibition effect was much profound when the ligand was IP injected at 250 μg/kg (left panel) or IV injected at 1000 μg/kg (right panel). No significant difference in body weight was observed among the four groups during the experiment period (FIG. 14B). After 30 days, the tumors averaged 5.5, 2.1, 3.5 and 2.1 grams in the Sham, MB109-IP250, MB109-IP1000 and MB109-IV1000 groups, respectively (FIG. 14C). The differences were visibly apparent (FIG. 14D).

The xenografted tumors of the Sham and MB109-IP250 groups were subjected to immuno-fluorescence analysis to visualize the protein expression of CD44. Significant reduction of CD44 expression was observed in the tumor tissue of the MB109 group as compared to the Sham group (FIG. 14E). Reduction of CD90 and AFP in the tumor tissue of MB109 group was also observed by immunohistochemistry analysis (FIGS. 14F and 14G, respectively). These results firmly support the conclusion that the remission of tumor growth resulted from the reduction of LCSC markers by MB109-triggered signaling pathways.

MB109 Treatment Leads to HBx⁺ Cell Cycle Arrest at G0/1.

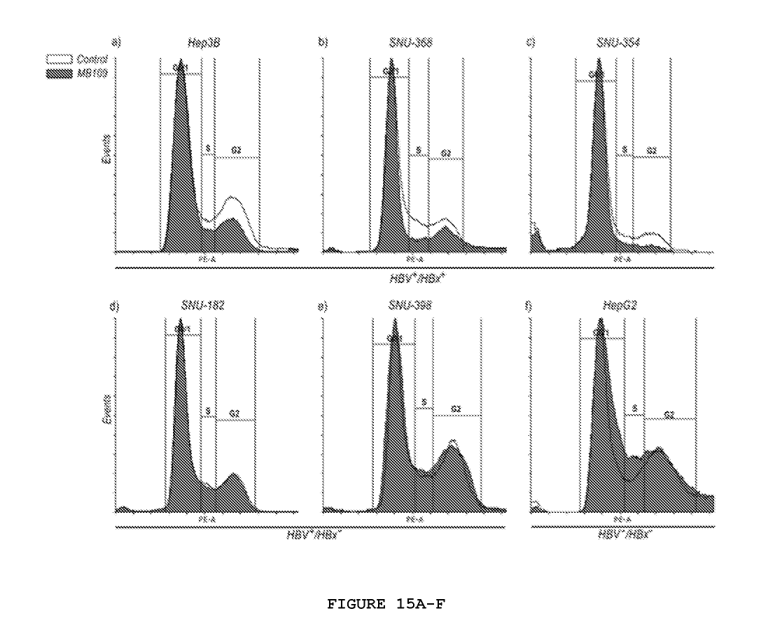

HBx disrupting the p53 function leads to uncontrolled cell cycle progression, causing loss of self-growth inhibition, which is a characteristic of cancer cells. MB109 revived the self-growth inhibition by signaling proteins. It was investigated to see if this phenomenon was due to a regulated cell cycle state. As a consequence of p21 overexpression and Survivin suppression, MB109 treatment induced HBV⁺/HBx⁺ HCC cells to arrest the cell cycle at the G0/1 phase (see FIGS. 15A, 15B and 15C). Cell cycle analysis revealed that when cells were treated with 200 ng/ml of MB109, there was a net increase of 8˜12% cells in G0/1 phase and a net decrease of 7˜13% cells in S/G2 phase (see TABLE 3).

TABLE 3

| | G0/1 | | S | | G2 | |
|---|---|---|---|---|---|---|
| | Control | MB109 | Control | MB109 | Control | MB109 |
| Hep3B | 62.9% | 75.2% | 7.0% | 5.6% | 30.1% | 19.2% |
| SNU-368 | 66.5% | 74.1% | 13.3% | 7.7% | 20.2% | 18.2% |
| SNU-354 | 79.1% | 89.1% | 6.8% | 4.8% | 14.1% | 6.1% |
| SNU-182 | 68.0% | 68.0% | 9.0% | 8.7% | 23.1% | 23.5% |
| SNU-398 | 54.1% | 55.4% | 11.2% | 11.0% | 34.7% | 33.6% |
| HepG2 | 55.5% | 56.8% | 10.5% | 13.2% | 34.1% | 30.0% |

MB109 treatment led to a reduction of Hep3B and SNU-354 cells in the G2 phase and a reduction of SNU-368 cells in the S phase. The difference seen in the cells is most likely due to SNU-368 proliferating much slower than Hep3B and SNU-354. Following the same trend, p21/survivin analysis demonstrated that the cell cycle of HBV⁺/HBx⁻ cells was not significantly affected by MB109 (see FIGS. 15D and 15E). For HepG2, a HBV⁻ cell line, MB109 slightly increased cells in S phase (˜3%) with a corresponding decrease in cells in the G2 phase (˜4%), and an unchanged G0/1 phase. While prolonged cell cycle arrest sometimes leads to apoptosis, there was no indication of apoptosis by using a DNA fragmentation analysis. From these results it was concluded that MB109 treatment arrests the cell cycle at G0/1 phase, which is closer to the natural state found in the liver.

MB109 is a Potent Inducer for Brown Adipogenesis.

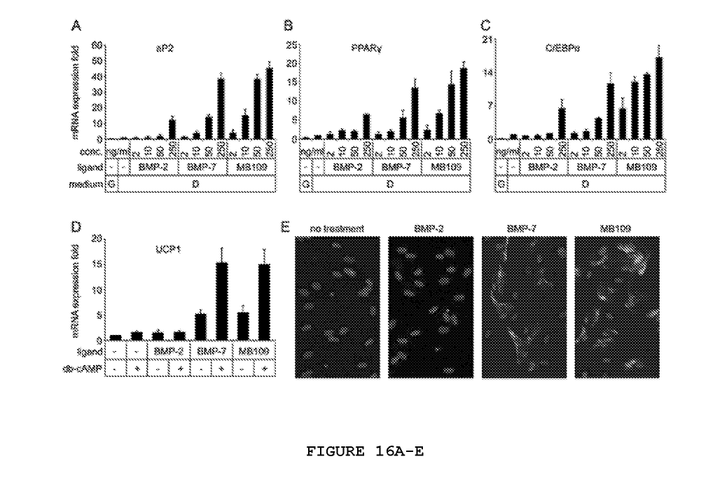

Capacity of MB109 to differentiate stem cells into brown adipocytes was compared with BMP-2 and BMP-7 in vitro using hASC. The mRNA expression levels of adipocyte protein 2 (aP2) in cells treated with BMP-2 were lower than those treated by BMP-7 or MB109 (see FIG. 16A). The mRNA levels of peroxisome proliferator-activated receptor (PPAR)γ and CCAAT-enhancer-binding proteins (C/EBP)α transcriptional activators necessary for adipogenesis, showed similar patterns as those of aP2 in cells treated by the ligands (see FIGS. 16B and 16C). Although these BMP ligands were all able to induce the expression of the adipogenesis markers in a dose dependent manner, MB109 was a better adipogenic factor than BMP-2 or BMP-7 at a lower dose, (i.e., less than 50 ng/ml). The mRNA expression of UCP1, a marker gene of brown adipocytes, was induced by BMP-7 as well as MB109, but not by BMP-2, and the levels of UCP1 mRNA were markedly enhanced by cAMP treatment (see FIG. 16D). The results indicate that the cells differentiated by MB109 possessed a verified thermogenic capacity as brown adipocytes. Expression of UCP1 was also analyzed at the protein level by immunocytochemistry. As shown in FIG. 16E, the capacity of MB109 to induce brown adipogenesis was comparable to that of BMP-7 (see FIG. 16E).

MB109 Suppresses Weight Gaining of High Fat Diet-Induced Obese Mice by Reducing Fat Mass.

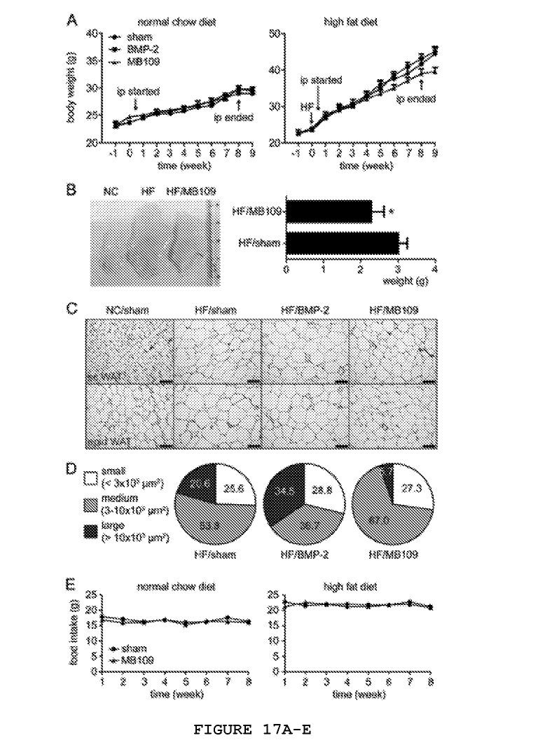

Effect of MB109 on brown adipogenesis was also analyzed using an animal model to determine whether systematic injection of MB109 could enhance brown adipogenesis and attenuate weight gain when mice were fed a high fat diet (i.e., induced obesity animal model). While intraperitoneal injection (200 μg/kg/week for 8 weeks) of MB109 to mice fed with a normal chow (NC) diet had no change in body weight, MB109 suppressed weight gain in mice fed a high fat (HF) diet (see FIG. 17A). However, the effect of MB109 on suppressing weight gain in mice fed a HF diet was not obvious until after 5 weeks of injection. In comparison, intraperitoneal injection of BMP-2 did not significantly affect weight gain in mice fed a HF diet (see FIG. 17A). The volume and weight of epididymal fat tissue from the HF/MB109 group was noticeably smaller than those from the HF/sham group (see FIG. 17B). Histological analysis of the adipose tissues showed that the size of individual adipocytes in the HF/MB109 group mice were smaller than those from the HF/sham group (see FIG. 17C). Percent distribution analysis of adipocyte sizes revealed that the adipocyte population in the epididymal fat tissues from the HF/MB109 group had shifted from large to medium size as compared with those from the HF/sham or HF/BMP-2 groups (see FIG. 17D). The results suggest that the smaller size of epididymal fat tissue from the HF/MB109 group is a direct result of the reduction in size of individual adipocytes. The volumes of BATs or H&E staining of cross sections of BATs showed little difference among groups. Injection of ligands did not cause any change in the food consumption of mice (see FIG. 17E) or behavioral performance.

MB109 Induces Browning of Subcutaneous White Adipose Tissue.

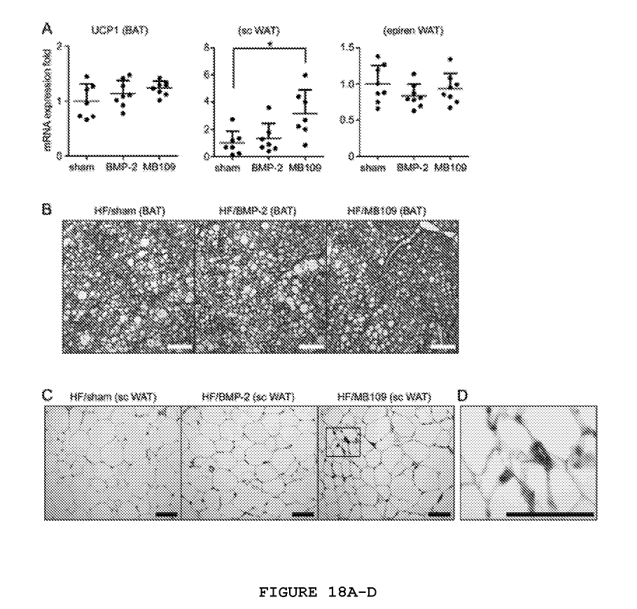

Real-time PCR analysis showed that the differences in mRNA expression of UCP1 in the BATs among groups fed with high fat diet were not statistically significant (see FIG. 18A). While UCP1 expression levels in the epirenal WATs showed no statistically significant difference among the groups, those in the subcutaneous WAT from the HF/MB109 group were higher than those of the HF/sham or HF/BMP-2 groups (see FIG. 18A). Consistent with results of real-time PCR analysis, immunohistochemical staining showed abundant expression of UCP1 protein in the BATs with little difference among groups (FIG. 18B). Expression of UCP1 protein in the subcutaneous WATs of HF/MB109 group was enhanced and detected in the area of small multilocular cells (see FIGS. 18C and 18D).

Expression Levels of Cell Death-Inducing DFFA-Like Effector (Cidea) have been Reported to be Associated with Energy Expenditure and Metabolic Rate.

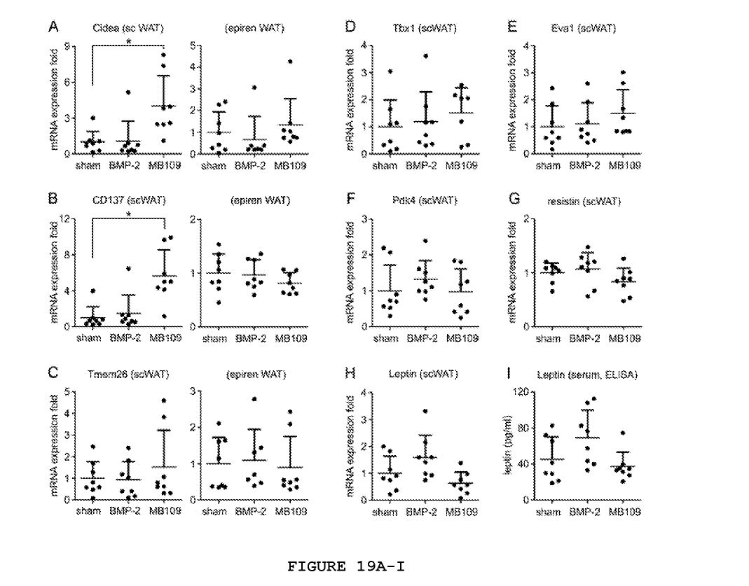

The mRNA expression level of Cidea was higher in the subcutaneous WAT from HF/MB109 group (see FIG. 19A, left). While there was a trend showing higher expression levels of Cidea in the epirenal WAT in the HF/MB109 group in comparison to the HF/sham group, the difference was not statistically significant (see FIG. 19A, right). In addition, the expression level of CD137, whose expression has been reported to be high in beige cells, was also elevated in the subcutaneous WATs of HF/MB109 group (see FIG. 19B, left), but not in the epirenal WATs (see FIG. 19B, right). However, the expression levels of Tmem26 and Tbx1, which have been reported to be beige adipocyte-selective genes, were not elevated in the WATs of the HF/MB109 group (see FIGS. 19C and 19D, respectively). Expression levels of Pdk4 and Eva1, which have been reported to be brown adipocyte-selective genes, were not elevated in the subcutaneous WAT of the HF/MB109 group (see FIGS. 19E and 19F).

Expression levels of adipokines associated with obesity and developing type 2 diabetes were also analyzed using real-time PCR and ELISA. Intraperitoneal injection of BMP-2 and MB109 showed little change in the tested groups for the mRNA expression levels of resistin (see FIG. 19G). In addition, differences in mRNA or protein expression of leptin between HF/sham and HF/MB109 groups were statistically not significant (see FIGS. 19H and 19I).

Periodical Injection of MB109 Attenuates Obesity-Mediated Increase of Blood Glucose Levels.

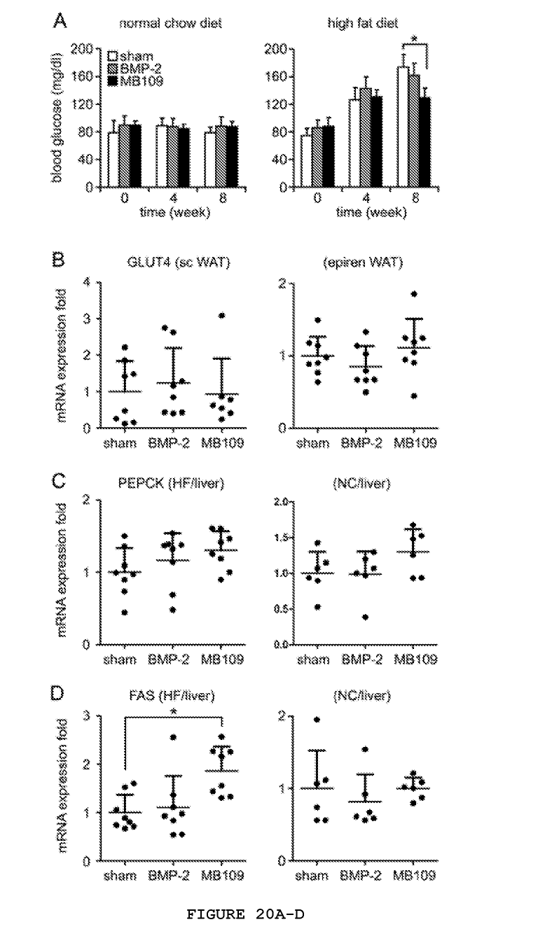

While blood glucose levels of mice fed with normal chow diet showed no difference for 8 weeks, those of the mice fed with high fat diet showed gradual increase in blood glucose levels in the HF/sham and HF/BMP-2 groups during the 8 weeks (see FIG. 20A). However, blood glucose levels of the HF/MB109 mice were lower than those of the HF/sham mice after 8 weeks of being fed a high fat diet (see FIG. 20A, right), suggesting that periodical injection of MB109 is able to attenuate the development of obesity-associated type 2 diabetes. Since obesity or type 2 diabetes have been reported to reduce gene expression of glucose transporter 4 (GLUT4) in the adipose tissues, we also analyzed GLUT4 mRNA expression in the subcutaneous and epirenal WATs. Periodical injection of BMP-2 or MB109 did not change mRNA expression levels of GLUT4 in the WATs from mice fed with high fat diet (see FIG. 20B).

Since BMP-9 has been reported to be highly expressed in the liver nonparenchyma cells and regulates expression of genes involved in glucose metabolism, mRNA expression patterns were analyzed for phosphoenolpyruvate carboxykinase (PEPCK) and fatty acid synthase (FAS) in the liver. Periodical injection of BMP-2 or MB109 (200 μg/kg/week) did change the overall morphology of the liver or the expression levels of PEPCK in the liver (see FIG. 20C). However, periodical injection of MB109, but not BMP-2, increased mRNA expression levels of FAS in the liver from mice fed with a HF diet, but not fed with a NC diet (see FIG. 20D, left and right, respectively).

A number of embodiments have been described herein. Nevertheless, it will be understood that various modifications may be made without departing from the spirit and scope of this disclosure. Accordingly, other embodiments are within the scope of the following claims.

1. A protein comprising two or more polypeptides having at least 90% sequence identity to SEQ ID NO:2 and having a methionine at position 1, wherein the protein has human bone morphogenic-9 (human BMP-9) activity.

2. The protein of claim 1, wherein the protein comprises a homodimer of two polypeptides each polypeptide comprising a sequence of SEQ ID NO:2, wherein the homodimer comprises a cysteine knot scaffold formed from three intra-molecular and one inter-molecular disulfide bonds.

3. The protein of claim 1, wherein the protein is expressed and isolated from bacteria selected from the group consisting of Escherichia Coli, Corynebacterium glutamicum, and Pseudomonas fluorescens.

4. The protein of claim 1, wherein the two or more polypeptides are encoded by a Escherichia Coli, Corynebacterium glutamicum, or Pseudomonas fluorescens codon optimized polynucleotide sequence having at least 85% sequence identity to SEQ ID NO:1 and which encodes a polypeptide of SEQ ID NO:2.

5. The protein of claim 4, wherein the two or more polypeptides are encoded by a Escherichia Coli codon optimized polynucleotide sequence comprising SEQ ID NO:1.

6. The protein of claim 1, wherein the protein has been isolated and refolded from a bacterial inclusion body.

7. The protein of claim 6, wherein the bacterial inclusion body is from Escherichia Coli.

8. The protein of claim 6, wherein the protein has been purified to remove bacterial host cell contaminants.

9. The protein of claim 6, wherein the protein has been re-folded using one or more of the following folding conditions:
a buffer pH between about 8.0 and 8.5;
a CHAPS concentration between about 2 and 4%;
a NaCl concentration between about 1 and 2 M;
a redox system having a GSH/GSSG molar ratio between about 5:1 and 1:2;
a protein concentration of about 0.2 mg/mL or lower; and
a refolding temperature/duration of about 4° C. for about 7 to 9 days.

10. A bioactive recombinant MB109 protein comprising SEQ ID NO:2 having bone morphogenetic protein-9 activity, wherein the MB109 protein is expressed and isolated from bacteria.

11. The bioactive recombinant MB109 of claim 10, wherein the protein comprises a homodimer of two polypeptide each comprising a sequence of SEQ ID NO:2.

12. The bioactive recombinant MB109 of claim 10, wherein the bacteria is selected from group consisting of Escherichia Coli, Corynebacterium glutamicum, or Pseudomonas fluorescens

13. The bioactive recombinant MB109 of claim 12, wherein the recombinant MB109 is expressed and isolated from an Escherichia Coli inclusion body.

14. The bioactive recombinant MB109 of claim 10, wherein the MB109 protein is encoded by a polynucleotide comprising at least 85% sequence identity to SEQ ID NO:1 and which polynucleotide encodes a polypeptide of SEQ ID NO:2.

15. The bioactive recombinant MB109 of claim 14, wherein the MB109 protein comprises a polypeptide encoded by a Escherichia Coli codon optimized polynucleotide sequence comprising SEQ ID NO:1.

16. The bioactive recombinant MB109 of claim 10, wherein the MB109 has been re-folded using one or more of the following folding conditions:
a buffer pH between about 8.0 and 8.5;
a CHAPS concentration between about 2 and 4%;
a NaCl concentration between about 1 and 2 M;
a redox system having a GSH/GSSG molar ratio between about 5:1 and 1:2;
a protein concentration of about 0.2 mg/mL or lower; and
a refolding temperature/duration of about 4° C. for about 7 to 9 days.

17. A pharmaceutical composition comprising the bioactive recombinant MB109 of claim 10 and a pharmaceutically acceptable carrier.

18. A method of treating cancer in a subject comprising administering the bioactive recombinant MB109 of claim 10.

19. The method of claim 18, wherein the cancer is selected from the group consisting of adrenocortical carcinoma, anal cancer, bladder cancer, brain tumors, and ependymomas, breast cancer, gastrointestinal carcinoid tumors, cervical cancer, colon cancer, endometrial cancer, esophageal cancer, extrahepatic bile duct cancer, ewings family of tumors (PNET), extracranial germ cell tumors, extragonadal germ cell tumors, eye cancer, including intraocular melanomas, gallbladder cancer, gastric cancer (stomach), gestational trophoblastic tumor, head and neck cancer, hypopharyngeal cancer, islet cell carcinoma, kidney Cancer (renal cell cancer), laryngeal cancer, leukemias, lip and oral cavity cancer, liver cancer, lung cancer, lymphomas, malignant mesothelioma, melanoma, merkel cell carcinoma, metasatic squamous neck cancer with occult primary, multiple myeloma and other plasma cell neoplasms, mycosis fungoides, myelodysplastic syndrome, myeloproliferative disorders, nasopharyngeal cancer, neuroblastoma, oral cancer, oropharyngeal cancer, osteosarcoma, ovarian epithelial cancer, ovarian germ cell tumor, pancreatic cancers, paranasal sinus and nasal cavity cancer, parathyroid cancer, penile cancer, pituitary cancer, plasma cell neoplasm, prostate cancer, rhabdomyosarcoma, rectal cancer, renal cell cancer (cancer of the kidney), transitional cell renal pelvis and ureter cancer, salivary gland cancer, sezary syndrome, skin cancers, small intestine cancer, soft tissue sarcoma, stomach cancer, testicular cancer, malignant thyoma, thyroid cancer, urethral cancer, uterine cancer, vaginal cancer, vulvar cancer, and Wilms' tumor.

20. The method of claim 19, wherein the cancer is liver cancer.

21-23. (canceled)

24. A method of treating obesity and/or an obesity associated disorder in a subject comprising administering the bioactive recombinant MB109 of claim 10.

25-29. (canceled)


SEQUENCE LISTING (36 sequences)

SEQ ID NO: 1 - DNA, 336 bp, Artificial Sequence
  MB109 coding sequence
atgtcagcaggtgctggttcgcattgtcaaaaaaccagtctgcgcgttaattttgaagatattggttgggactcctggattatcgcgccgaaagaatatgaagcctacgaatgcaaaggtggctgctttttcccgctggcagatgacgtcacgccgaccaaacatgctattgtccagacgctggtgcacctgaaattcccgaccaaagtgggcaaagcgtgctgtgttccgaccaaactgtctccgatcagtgtgctgtataaagatgacatgggtgtcccgaccctgaaataccattacgaaggcatgagtgtggcggaatgtggctgtcgctaa

SEQ ID NO: 2 - protein, 111 aa, Artificial Sequence
  Synthetic Construct
MSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 3 - DNA, 20 bp, Artificial Sequence
  Beta Actin Forward Primer
ctaaggccaaccgtgaaaag

SEQ ID NO: 4 - DNA, 20 bp, Artificial Sequence
  Beta Actin Reverse Primer
accagaggcatacagggaca

SEQ ID NO: 5 - DNA, 22 bp, Artificial Sequence
  p21 Forward Primer
ccagcctctggcattagaatta

SEQ ID NO: 6 - DNA, 22 bp, Artificial Sequence
  p21 Reverse Primer
cgggatgaggaggctttaaata

SEQ ID NO: 7 - DNA, 22 bp, Artificial Sequence
  Survivin Forward Primer
gatgacgaccccatagaggaac

SEQ ID NO: 8 - DNA, 24 bp, Artificial Sequence
  Survivin Reverse Primer
gggttaattcttcaaactgcttct

SEQ ID NO: 9 - DNA, 20 bp, Artificial Sequence
  HBx Forward Primer
acggggcgcacctctcttta

SEQ ID NO: 10 - DNA, 20 bp, Artificial Sequence
  HBx Reverse Primer
tgcctacagcctcctagtac

SEQ ID NO: 11 - DNA, 20 bp, Artificial Sequence
  AP2 Forward Primer
catgtgcagaaatgggatgg

SEQ ID NO: 12 - DNA, 20 bp, Artificial Sequence
  AP2 Reverse Primer
aacttcagtccaggtcaacg

SEQ ID NO: 13 - DNA, 23 bp, Artificial Sequence
  Cyclophilin Forward Primer
cgaggaaaaccgtgtactattag

SEQ ID NO: 14 - DNA, 19 bp, Artificial Sequence
  Cyclophilin Reverse Primer
tgctgtctttgggaccttg

SEQ ID NO: 15 - DNA, 17 bp, Artificial Sequence
  C/EBP1-alpha Forward Primer
tggacaagagcaacgag

SEQ ID NO: 16 - DNA, 20 bp, Artificial Sequence
  C/EBP1-alpha Reverse Primer
tcattgtcactggtcagctc

SEQ ID NO: 17 - DNA, 20 bp, Artificial Sequence
  PPAR gamma Forward Primer
gagcccaagtttgagtttgc

SEQ ID NO: 18 - DNA, 22 bp, ARtificial Sequence
  PPAR gamma Reverse Primer
gcaggttgtcttgaatgtcttc

SEQ ID NO: 19 - DNA, 21 bp, Artificial Sequence
  CD137 Forward Primer
cctgtgataactgtcagcctg

SEQ ID NO: 20 - DNA, 22 bp, Artificial Sequence
  CD137 Reverse Primer
tcttgaacctgaaatagcctgc

SEQ ID NO: 21 - DNA, 21 bp, Artificial Sequence
  CIDEA Forward Primer
atcacaactggcctggttacg

SEQ ID NO: 22 - DNA, 21 bp, Artificial Sequence
  CIDEA Reverse Primer
tactacccggtgtccatttct

SEQ ID NO: 23 - DNA, 19 bp, Artificial Sequence
  Cyclophilin Forward Primer
cagacgccactgtcgcttt

SEQ ID NO: 24 - DNA, 23 bp, Artificial Sequence
  Cyclophilin Reverse Primer
tgtctttggaactttgtctgcaa

SEQ ID NO: 25 - DNA, 20 bp, Artificial Sequence
  FAS Forward Primer
cccctctgttaattggctcc

SEQ ID NO: 26 - DNA, 20 bp, Artificial Sequence
  FAS Reverse Primer
ttgtggaagtgcaggttagg

SEQ ID NO: 27 - DNA, 20 bp, Artificial Sequence
  GLUT4 Forward Primer
gattctgctgcccttctgtc

SEQ ID NO: 28 - DNA, 20 bp, Artificial Sequence
  GLUT4 Reverse Primer
attggacgctctctctccaa

SEQ ID NO: 29 - DNA, 21 bp, Artificial Sequence
  Leptin Forward Primer
gtgcctatccagaaagtccag

SEQ ID NO: 30 - DNA, 20 bp, Artificial Sequence
  Leptin Reverse Primer
tgaagcccaggaatgaagtc

SEQ ID NO: 31 - DNA, 21 bp, Artificial Sequence
  PEPCK Forward Primer
ccatcccaactcgagattctg

SEQ ID NO: 32 - DNA, 21 bp, Artificial Sequence
  PEPCK Reverse Primer
ctgagggcttcatagacaagg

SEQ ID NO: 33 - DNA, 21 bp, Artificial Sequence
  Resistin Forward Primer
tccttttcttccttgtccctg

SEQ ID NO: 34 - DNA, 21 bp, Artificial Sequence
  Resistin Reverse Primer
gactgtccagcaatttaagcc

SEQ ID NO: 35 - DNA, 21 bp, Artificial Sequence
  TMEM26 Forward Primer
gcaccatcactagagaccaac

SEQ ID NO: 36 - DNA, 20 bp, Artificial Sequence
  TMEM26 Reverse Primer
acaagaatgccagagaccag